(19) United States
(12) Patent Application Publication
(10) Pub. No.: US 2003/0130180 A1
(43) Pub. Date: Jul. 10, 2003

(54) Utilization of an aminopeptidase inhibitor

(76) Inventor: Walter Schubert, Biederitz (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 09/937,191

(22) PCT Filed: Jan. 24, 2001
(86) PCT No.: PCT/EP01/00746

(30) Foreign Application Priority Data
Jan. 24, 2000 (DE) 100028209

(51) Int. Cl. A61K 38/17

(52) U.S. Cl. 514/12

(57) ABSTRACT

The invention concerns the utilization of at least one aminopeptidase inhibitor for the production of a medicament used in the treatment of tumor diseases and/or immune diseases, whereby the at least one aminopeptidase inhibitor causes blocking of polarization of invasive human or animal tumor and/or immune cells by modifying at least one surface protein CD13 as member of a protein network on the surface of the tumor and/or immune cells, whereby the protein network comprises up to 30 surface proteins from a defined group. The invention also concerns a pharmaceutical preparation and a method for identifying at least one additional inhibitor acting in combination with the at least one aminopeptidase inhibitor.

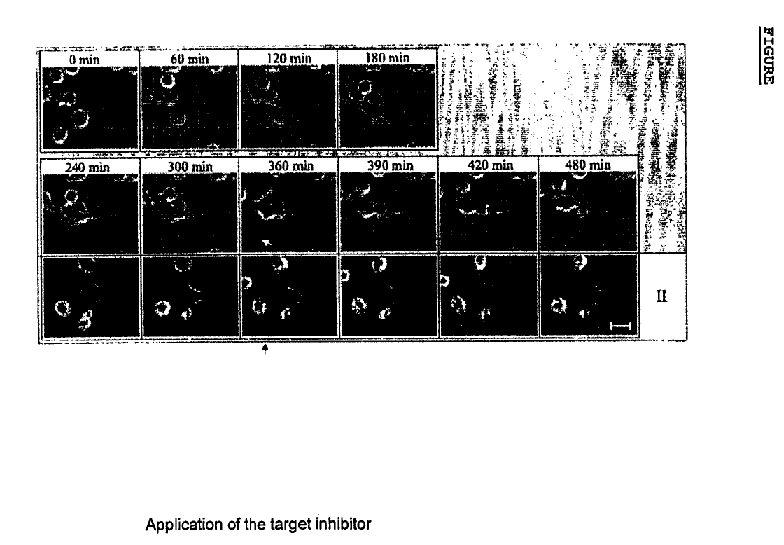

The present invention relates to a utilization of at least one aminopeptidase inhibitor for the production of a medicament used in the treatment of tumor diseases and/or immune diseases, a corresponding pharmaceutical preparation, a method for identifying at least one aminopeptidase inhibitor and a method for identifying at least one additional inhibitor acting in combination with the at least one aminopeptidase inhibitor.

Aminopeptidases are cell surface enzymes which split peptides. They are expressed by different types of cells. Their molecular function is, amongst others, the degradation of biologically active peptides. Additional physiological functions of aminopeptidases, in particular their cellular functions, have not been fully established as yet. Recent research has shown that aminopeptidase inhibitors are capable of suppressing the proliferation rate and the invasion of tumor cells. This suppression of the invasion was generally believed to be a result of the proteolytic activity of cell-surface-associated aminopeptidases which split the extracellular matrix proteins, thus allowing the tumor cells to enter organs and migrate within them. Some of the known aminopeptidase inhibitors are actinonin, bestatin as well as potent inhibitors of the homophtalimide type.

According to J. Yoneda et al., in: Clin. Exp. Metastasis 10, 49-59, 1992, bestatin is capable of preventing degradation of the type IV collagen, thus also preventing the invasion of tumor cells. It is further disclosed in this prior art publication that bestatin does not have any influence on tumor cell adhesion or on the migration to the extracellular matrix.

In a publication in Biol. Pharm. Bull. 22, 1010-1012, 1999, it is disclosed that the inhibition of the invasion by means of an aminopeptidase inhibitor of the homophtalimide type PIQ-22 is due to a suppression of the formation of cell extensions, with the cause of such suppression remaining unclear, and an inhibition of the aminopeptidase N, in the following also referred to as CD13, by PIQ-22 is considered impossible. According to this prior art publication, it was shown in in vitro experiments using an unspecific matrigel analytic system that the two aminopeptidase inhibitors actinonin and bestatin which will inhibit CD13 do not have an effect on tumor cell invasion. For bestatin, no effect on the formation of cell extensions was detected, either.

What is particularly disadvantageous is the fact that the examination methods existing so far will not render the in vitro conditions, thus frequently leading to unsatisfactory results, for which reason the effect of the individual aminopeptidase inhibitors cannot be established in detail. Usually, it remains unclear whether inhibition of the aminopeptidases in the case of tumors will be effective in vivo, i.e. in a patient, and whether for certain kinds of tumors, the inhibition of aminopeptidases might actually even result in an adversary effect, consisting in a potentiation of the invasive behavior in vivo.

Furthermore, the effective mechanism in the aminopeptidase inhibitors of known effect is totally unknown for which reason no new substances can be developed or identified which are capable of acting very specifically only because they interfere with the cell functions in a known manner. For example, it has not been possible to ascertain with which other proteins aminopeptidases will interact in one and the same cell and in which way such interaction will code for complex cell functions. Consequently, it is not known either whether and which cellular mechanisms based on such interaction might be blocked specifically by an inhibition of the aminopeptidases and which new indications may result to therefrom for a clinical use of such inhibitors or any substances further developed from such inhibitors.

It is, therefore, the object of the present invention to provide an aminopeptidase inhibitor of predefined and controllable effective behavior which can be used for the production of a medication used in the treatment of tumor diseases and/or immune diseases. It is another object of the present invention to provide a corresponding pharmaceutical preparation, a method for identifying at least one such aminopeptidase inhibitor as well as a method for identifying at least one additional inhibitor acting in combination with the at least one aminopeptidase inhibitor.

One of these objects is accomplished by a utilization of at least one aminopeptidase inhibitor for the production of a medication used in the treatment of tumor diseases and/or immune diseases whereby the at least, one aminopeptidase inhibitor causes blocking of polarization of invasive human or animal tumor and/or immune cells by modifying at least one surface protein CD13 as member of a protein network on the surface of the tumor and/or immune cells, whereby said protein network comprises up to 30 surface proteins from a group consisting of

```
  1. CD4
  2. CD8
  3. HLA-DR
  4. HLA-DQ
  5. CD3
  6. CD26
  7. CD38
  8. CD45RA
  9. CD16
10. CD57
11. CD56
12. CD7
13. CD54
14. CD58
15. CD138
16. CD13
17. CD62L
18. CD71
19. CD11b
20. CD36
21. CD29
22. CD49d
23. CD18
24. CD49f
25. CD19
26. CD2
27. CD20
28. CD10
29. CD44
30. CD80.
```

By means of a method of simultaneously detecting a number of cell surface proteins, it has been established that aminopeptidases will control cell surface proteins which are not part of the class of proteolytic enzymes, but belong to the class of the adhesion molecules, which adhesion molecules—in a certain combination and geometric array—will be decisive for the polarization of the cells. Consequently, aminopeptidases appear to be superordinate control proteins in a protein network consisting of up to 30 different cell surface protein species which—through specific interaction with one another—will control polarization of tumor cells and other invasive cells such as immune cells and which are listed above. The inhibition of at least one aminopeptidase will lead to a reproducible modification of surface protein combinations on the cell surface which will always also involve a modification of CD13. It has been shown in cell-biological experiments with tumor cells and immune cells that the inhibition and the associated modification of the surface protein combinations resulted in a complete blocking of the polarization of the tumor cells or immune cells. The term polarization as used here shall denote a process in which a primarily spherical cell will transition into an oblong, elongated cell shape, via various intermediate states. This process which constitutes a change of shape controlled by the complex protein network is the prerequisite for cell migration, since only cells of oblong shape are capable of migrating. The polarization process therefore needs to precede all cell migration processes, including invasion.

The invention is therefore based on the finding that the very aminopeptidase inhibitors which will cause modification of at least the surface protein CD13 as member of the specific, above-defined protein network of up to 30 surface proteins, will quite specifically inhibit the first and hence the most important step in the invasion and thus be suitable for use in the production of an extremely specifically acting and thus extremely effective medication for treating tumor diseases and/or immune diseases.

The at least one aminopeptidase inhibitor may e.g. be an aminopeptidase inhibitor of the homophtalimide type and/or actinonin and/or bestatin and/or an antibody, in particular a monoclonal antibody against one of the surface proteins. Bestatin in particular acts through the said effective mechanism, against the assumptions set out above, leading to a modification of the surface proteins of the protein network which comprises proteins from the above mentioned group. Furthermore, actinonin, RB 3014 and a monoclonal antibody (clone SJ1D1) directed against an extracellular domain of CD13 have shown to be particularly effective.

Since the above identified aminopeptidase inhibitors, besides allowing the polarization of tumor cells, also effectively aid to suppress the polarization of immune cells, use of the aminopeptidase inhibitors will allow the preparation of effective medications for treating autoimmune diseases or rejections of transplanted organs or allergies, in particular allergies of the respiratory tract.

Advantageously, at least one additional inhibitor may be used for the production of the medication, which inhibitor will modify and/or inhibit at least one surface protein that is not an aminopeptidase. The term inhibition in this case shall refer to the general inhibition of the function of the at least one surface protein which may also be brought about by an expression modification. By using an inhibitor combination, blocking of the polarization may be increased enormously. For example, an antibody against CD45RA may be used as an additional inhibitor. This very inhibitor will especially increase the effect of an aminopeptidase inhibitor as defined above, thus allowing the polarization to be inhibited in a specific and particularly effective manner by means of this inhibitor combination.

Especially, besides the modification of CD13, at least one aminopeptidase inhibitor and/or at least one additional inhibitor may cause a modification of at least one further surface protein of the tumor cells and/or immune cells which is responsible for adhesion to endothelial cells and/or extracellular structures, in particular to organ-specific endothelial cells and/or to organ-specific extracellular structures. At least one aminopeptidase inhibitor and/or at least one additional inhibitor may also cause a modification of the adhesive functions of endothelial cells. In this way, any binding of the tumor cells and/or immune cells to the endothelial cells can be prevented, which is imperative to the polarization. In order to specifically prevent an invasion of a certain organ or migration within such organ, those aminopeptidase inhibitors or additional inhibitors may be used which will specifically block any binding to the organ-specific endothelial cells and/or the organ-specific extracellular structures.

It is furthermore considered-particularly advantageous if the expression of at least one surface protein, in particular an adhesion molecule, can be influenced by at least one aminopeptidase inhibitor and/or by at least one additional inhibitor.

One of the objects set out above is accomplished by a pharmaceutical preparation which can be produced using at least one aminopeptidase inhibitor and/or a combination of at least one aminopeptidase inhibitor and at least one additional inhibitor as described above.

Furthermore, one of the above-mentioned objects is accomplished by a method for identifying aminopeptidase inhibitors which will cause blocking of polarization of invasive human or animal tumor and/or immune cells, in which method surface protein combinations of a protein network are first detected which are on the surface of the untreated tumor cells and/or immune cells, whereby the protein network comprises up to 30 surface proteins from a group comprising

```
  1. CD4
  2. CD8
  3. HLA-DR
  4. HLA-DQ
  5. CD3
  6. CD26
  7. CD38
  8. CD45RA
  9. CD16
10. CD57
11. CD56
12. CD7
13. CD54
14. CD58
15. CD138
16. CD13
17. CD62L
18. CD71
19. CD11b
20. CD36
21. CD29
22. CD49d
23. CD18
24. CD49f
25. CD19
26. CD2
27. CD20
28. CD10
29. CD44
30. CD80.
```

In a next step, these or similar tumor cells and/or immune cells are treated with at least one aminopeptidase inhibitor. Subsequently, the surface protein combinations of the protein network which are on the surface of the treated tumor cells and/or immune cells are detected and compared with the surface protein combinations of the protein network which are on the surface of the untreated tumor cells and/or immune cells. If there is a divergence in that there is at least one modification of the surface protein CD13, the at least one aminopeptidase inhibitor will cause blocking of polarization of the tumor cells and/or immune cells.

In an additional step, the at least one identified aminopeptidase inhibitor may be added to at least one polarizing tumor cell and/or immune cell, and the further development of the at least one polarizing tumor cell and/or immune cell may be detected in order to thus prove the actual blocking of polarization.

The method may furthermore comprise a control step in which binding of the untreated tumor cells and/or immune cells to organ-specific endothelial cells and/or organ-specific extracellular structures is detected, binding of the tumor cells and/or immune cells treated with the at least one identified aminopeptidase inhibitor to the organ-specific endothelial cells and/or the organ-specific extracellular structures is detected and the detected bindings are compared. If reduced binding is detected in case of the treated tumor cells and/or immune cells, polarization will be inhibited in a particularly effective manner since an effective organ-specific adhesion will be prevented.

One of the above objects is accomplished by a method for identifying inhibitors which will cause blocking of polarization of invasive human or animal tumor cells and/or immune cells, acting in combination with at least one aminopeptidase inhibitor, in which method surface protein combinations of a protein network which are on the surface of the untreated tumor cells and/or immune cells, are first of all detected, whereby the protein network comprises up to 30 surface proteins from a group of the composition already set out above. These or similar tumor cells and/or immune cells are treated with at least one potential inhibitor, and the surface protein combinations of the protein network which are on the surface of the treated tumor cells and/or immune cells are detected. Subsequently, the detected surface protein combinations are compared, and, if there is a divergence in that there is at least one modification of a surface protein, the at least one inhibitor will be suitable for blocking polarization of the tumor cells and/or immune cells.

In addition to being treated with the at least one inhibitor, the or the identical tumor cells and/or immune cells may also be treated with at least one aminopeptidase inhibitor, whereby the combination of the at least one inhibitor and the at least one aminopeptidase inhibitor will cause blocking of polarization of the tumor cells and/or immune cells, if there is a divergence in the surface protein combinations detected in the two steps in that there is at least one modification of a surface protein CD13.

The method may furthermore comprise another step in which the at least one identified inhibitor or a combination of the at least one identified inhibitor and the at least one aminopeptidase inhibitor is added to at least one polarizing tumor cell and/or immune cell, and the further development of the at least one polarizing tumor cell and/or immune cell is detected.

It is considered advantageous if the method comprises a control step in which binding of the untreated tumor cells and/or immune cells to organ-specific endothelial cells and/or to organ-specific extracellular structures is detected, in which binding of the tumor cells and/or immune cells which were treated with the at least one identified inhibitor and/or with a combination of the at least one identified inhibitor and the at least one aminopeptidase inhibitor, to the organ-specific endothelial cells and/or to the organ-specific extracellular structures is detected, and in which the detected bindings are compared.

Principally, detecting of the surface protein combinations may comprise procedural steps of an automated method for determining molecular classes, molecular groups or molecular parts in a solid or liquid object according to DE 197 09 348 C. In these steps, surface proteins may be examined and measured in one and the same object, i.e. in a sample of immune cells and/or tumor cells for example, by sequentially applying reagent solutions Yn (n=1,2,3, . . . N) by means of an automated apparatus, said procedural steps being:

I. taking a first reagent solution Y1 from a vessel containing the reagent solution,

II. applying said reagent solution Y1 to the object which is on an object slide,

III. allowing the reagent solution to react for an automatically set period of time,

IV. recording at least one individual labeling pattern of the object previously labeled with said first reagent solution Y1,

V. repeating steps I-IV by applying said first reagent solution Y1 or a second reagent solution Y2 or a mixture of said first and second reagent solutions, and

VI. repeating steps I to V with further reagent solutions Yn (n=2,3, . . . N) or a mixture thereof and whereby

the labeling distribution patterns obtained in each cycle of the method are turned into a complex molecular combination pattern of the object to be examined by computer-aided image overlay.

From this combination pattern, information may be gained on the presence of the above mentioned proteins, thus also allowing detection of the surface protein combinations, if the reagent solutions used contain labeled substances directed against the proteins in question.

In the control steps listed above, it is checked whether polarization is prevented by the at least one aminopeptidase inhibitor and/or by the at least one additional inhibitor by inhibiting any binding of certain molecules to defined structures. These control steps can be carried out by passing immune cells (lymphocytes) and/or tumor cells in the form of a continuous cell flow in a special apparatus described in DE 199 32 158 A over at least one sample with the defined structures. While, if the cells were not treated with the at least one aminopeptidase inhibitor and/or the at least one additional inhibitor, the cells should bind to the defined structures, after treatment of the cells with the at least one aminopeptidase inhibitor and/or the at least one additional inhibitor, however, there will not be any binding, or reduced binding only, to said structures. The sample may for example consist of an organ tissue section.

Further features and advantages of the invention may be gathered from the examination results listed in the following, which are described with reference to a drawing figure, amongst others.

The (only) FIGURE shows a time sequence of photographic images of polarizing cells, untreated as well as treated with a target inhibitor.

In numerous examinations, it was possible to identify the 30 cell surface proteins already listed above which belong to a specific protein network controlling the early polarization stages of tumor cells and immune cells. In a further examination, surface protein combinations of this protein network of Karpas cells were detected, both in untreated form and after treatment with the aminopeptidase inhibitor actinonin. For this purpose, two groups V1 and V2 of Karpas cells were formed, with the cells of group Vl remaining untreated, while the cells of group V2 were treated with actinonin. Table 2 lists those surface protein combinations which are present both in the actinonin-treated cells and in the untreated cells. However, this table and the further tables 3 and 4 only give examples of 18 of the 30 proteins, with the detected proteins being designated 1 and the non-detected proteins being designated 0.

The proteins are continuously numbered 1 to 18, with the nomenclature being notable from table 1.

TABLE 1

```
  1. CD2
  2. CD3
  3. CD4
  4. CD8
  5. CD16
  6. CD56
  7. CD57
  8. CD26
  9. CD38
10. CD71
11. HLA-DR
12. HLA-DQ
13. CD11b
14. CD45RA
15. CD7
16. CD62L
17. CD36
18. CD19
```

The cell numbers stated in tables 2 to 4 refer to each 1,000 cells examined in groups V1 and V2. Table 2 lists a total number of 203 different protein combinations.

Table 3 lists the surface protein combinations which only occur in the untreated Karpas cells and are never found in the actinonin-treated Karpas cells. The number of protein combinations listed in this table 3 amounts to 131.

Finally, table 4 exclusively lists those surface protein combinations which occur exclusively in the actinonin-treated Karpas cells. Table 4 contains 60 different protein combinations.

It may be gathered from tables 2 to 4 that, if one examines merely 18 proteins of the 30 proteins, a total of 394 different surface protein combinations will occur, with a total of 334 different combinations occurring in the untreated cells and a total of 263 different combinations being detected in the treated cells. The modification of the surface protein combinations thus detected results in a specific blocking of cell polarization.

A further examination is explained with reference to the only FIGURE. (I) shows the normal cellular process of tumor cell polarization. By in vitro life imaging it is recorded how a sarcoma cell polarizes from a primarily spherical cell shape, forming 3 cell extensions (tripolar cell shape) and subsequently specific involution of only one of said three extensions (white arrow at 360 min). The definition of a longitudinal axis is a prerequisite for the subsequent cell migration. It is shown in (II) that the application of a selective target inhibitor, in this case a monoclonal antibody, against an extracellular domain of CD13 (black arrow) will completely prevent cell polarization. The cell will become spherical and highly adhesive, which is notable from a comparison of a photographic image of the inhibited cell (II) after 480 min and a photographic image of the non-inhibited cell (I) after 480 min (I).

Similar results are obtained if actinonin or bestatin are used as target inhibitors.

TABLE 2

| | | | | | | | | | | | | | | | | | | | V1 | V2 |
|---|---|---|---|---|---|---|---|---|---|---|---|---|---|---|---|---|---|---|---|---|
| | | | | | | | | | | | | | | | | | | | no. of | no. of |
| | | | | | | | | | | | | | | | | | | | cells | cells |
| Protein code¹ | Proteins [1-18], binary | | | | | | | | | | | | | | | | | | In | In |
| No. | 1 | 2 | 3 | 4 | 5 | 6 | 7 | 8 | 9 | 10 | 11 | 12 | 13 | 14 | 15 | 16 | 17 | 18 | 1/1000 | 1/1OOO |
| 1 | 0 | 0 | 0 | 0 | 0 | 0 | 0 | 0 | 0 | 0 | 0 | 0 | 0 | 0 | 0 | 0 | 0 | 0 | 136.4 | 153.2 |
| 2 | 0 | 0 | 1 | 0 | 0 | 0 | 0 | 1 | 0 | 1 | 0 | 1 | 0 | 0 | 1 | 0 | 0 | 0 | 78.2 | 91.9 |
| 3 | 0 | 0 | 1 | 0 | 0 | 0 | 0 | 1 | 0 | 0 | 0 | 1 | 0 | 0 | 0 | 0 | 0 | 0 | 71.9 | 57.6 |
| 4 | 0 | 0 | 0 | 0 | 0 | 0 | 0 | 1 | 0 | 0 | 0 | 1 | 0 | 0 | 0 | 0 | 0 | 0 | 40.0 | 62.2 |
| 5 | 0 | 0 | 1 | 0 | 0 | 0 | 0 | 1 | 0 | 0 | 0 | 1 | 0 | 0 | 1 | 0 | 0 | 0 | 50.7 | 37.9 |
| 6 | 0 | 0 | 1 | 0 | 0 | 0 | 0 | 1 | 0 | 1 | 0 | 1 | 0 | 0 | 0 | 0 | 0 | 0 | 43.9 | 44.3 |
| 7 | 0 | 0 | 1 | 1 | 0 | 0 | 0 | 1 | 0 | 1 | 0 | 1 | 0 | 0 | 1 | 0 | 0 | 0 | 43.0 | 37.6 |
| 8 | 0 | 0 | 0 | 0 | 0 | 0 | 0 | 1 | 0 | 0 | 0 | 0 | 0 | 0 | 0 | 0 | 0 | 0 | 26.5 | 41.8 |
| 9 | 0 | 0 | 1 | 0 | 0 | 0 | 0 | 1 | 0 | 1 | 1 | 1 | 0 | 0 | 1 | 0 | 0 | 0 | 33.2 | 31.4 |
| 10 | 0 | 0 | 1 | 1 | 0 | 0 | 0 | 1 | 0 | 1 | 1 | 1 | 0 | 1 | 1 | 0 | 0 | 0 | 23.5 | 29.7 |
| 11 | 0 | 0 | 0 | 0 | 0 | 0 | 0 | 0 | 0 | 0 | 0 | 1 | 0 | 0 | 0 | 0 | 0 | 0 | 20.6 | 29.3 |
| 12 | 0 | 0 | 1 | 1 | 0 | 0 | 0 | 1 | 0 | 1 | 1 | 1 | 0 | 0 | 1 | 0 | 0 | 0 | 21.5 | 24.2 |
| 13 | 0 | 0 | 1 | 0 | 0 | 0 | 0 | 1 | 0 | 0 | 1 | 1 | 0 | 0 | 1 | 0 | 0 | 0 | 29.4 | 15.4 |
| 14 | 0 | 0 | 0 | 0 | 0 | 0 | 0 | 1 | 0 | 1 | 0 | 1 | 0 | 0 | 0 | 0 | 0 | 0 | 13.2 | 22.3 |
| 15 | 1 | 0 | 1 | 1 | 0 | 0 | 0 | 1 | 0 | 1 | 1 | 1 | 0 | 1 | 1 | 0 | 0 | 0 | 15.1 | 17.3 |
| 16 | 0 | 0 | 1 | 0 | 0 | 0 | 0 | 1 | 0 | 1 | 1 | 1 | 0 | 1 | 1 | 0 | 0 | 0 | 17.1 | 15.0 |
| 17 | 0 | 0 | 1 | 0 | 0 | 0 | 0 | 1 | 0 | 1 | 0 | 1 | 0 | 1 | 1 | 0 | 0 | 0 | 15.7 | 15.5 |
| 18 | 0 | 0 | 1 | 1 | 0 | 0 | 0 | 1 | 0 | 1 | 0 | 1 | 0 | 1 | 1 | 0 | 0 | 0 | 14.6 | 9.7 |
| 19 | 0 | 0 | 0 | 0 | 0 | 0 | 0 | 1 | 0 | 1 | 0 | 1 | 0 | 0 | 1 | 0 | 0 | 0 | 9.8 | 13.4 |
| 20 | 0 | 0 | 0 | 0 | 0 | 0 | 0 | 0 | 0 | 1 | 0 | 0 | 0 | 0 | 0 | 0 | 0 | 0 | 3.7 | 14.5 |
| 21 | 0 | 0 | 1 | 1 | 0 | 0 | 0 | 1 | 0 | 0 | 0 | 1 | 0 | 0 | 1 | 0 | 0 | 0 | 11.1 | 6.3 |
| 22 | 0 | 0 | 1 | 0 | 0 | 0 | 0 | 1 | 0 | 0 | 1 | 1 | 0 | 0 | 0 | 0 | 0 | 0 | 9.2 | 6.7 |
| 23 | 0 | 0 | 1 | 0 | 0 | 0 | 0 | 0 | 0 | 0 | 0 | 1 | 0 | 0 | 0 | 0 | 0 | 0 | 7.5 | 7.4 |
| 24 | 0 | 0 | 1 | 0 | 0 | 0 | 0 | 1 | 0 | 0 | 0 | 0 | 0 | 0 | 0 | 0 | 0 | 0 | 8.6 | 5.7 |
| 25 | 0 | 0 | 0 | 0 | 0 | 0 | 0 | 0 | 0 | 0 | 0 | 0 | 0 | 0 | 1 | 0 | 0 | 0 | 6.9 | 7.3 |
| 23 | 0 | 0 | 0 | 0 | 0 | 0 | 0 | 0 | 0 | 1 | 0 | 1 | 0 | 0 | 0 | 0 | 0 | 0 | 3.1 | 11.0 |
| 27 | 0 | 1 | 0 | 0 | 0 | 0 | 0 | 0 | 1 | 1 | 0 | 0 | 0 | 0 | 0 | 0 | 0 | 0 | 5.9 | 7.5 |
| 28 | 0 | 0 | 0 | 0 | 0 | 0 | 0 | 1 | 0 | 1 | 0 | 0 | 0 | 0 | 0 | 0 | 0 | 0 | 4.4 | 8.9 |
| 29 | 0 | 0 | 0 | 0 | 0 | 0 | 0 | 1 | 0 | 0 | 0 | 1 | 0 | 0 | 1 | 0 | 0 | 0 | 4.9 | 7.0 |
| 30 | 0 | 0 | 1 | 1 | 0 | 0 | 0 | 1 | 0 | 0 | 1 | 1 | 0 | 0 | 1 | 0 | 0 | 0 | 7.7 | 4.0 |
| 31 | 1 | 0 | 1 | 0 | 0 | 0 | 0 | 1 | 0 | 1 | 1 | 1 | 0 | 1 | 1 | 0 | 0 | 0 | 6.8 | 2.8 |
| 32 | 0 | 0 | 0 | 0 | 0 | 0 | 0 | 0 | 1 | 0 | 0 | 0 | 0 | 0 | 0 | 0 | 0 | 0 | 3.5 | 6.1 |
| 33 | 0 | 0 | 1 | 0 | 0 | 0 | 0 | 1 | 0 | 1 | 1 | 1 | 0 | 0 | 0 | 0 | 0 | 0 | 4.2 | 5.3 |
| 34 | 0 | 0 | 1 | 0 | 0 | 0 | 0 | 1 | 0 | 0 | 1 | 1 | 0 | 1 | 1 | 0 | 0 | 0 | 6.2 | 3.2 |
| 35 | 0 | 0 | 1 | 0 | 0 | 0 | 0 | 1 | 0 | 0 | 0 | 1 | 0 | 1 | 1 | 0 | 0 | 0 | 4.5 | 4.8 |
| 38 | 0 | 0 | 0 | 0 | 0 | 0 | 0 | 0 | 1 | 1 | 0 | 0 | 0 | 0 | 0 | 0 | 0 | 0 | 3.0 | 5.4 |
| 37 | 0 | 1 | 0 | 0 | 0 | 0 | 0 | 0 | 1 | 1 | 0 | 0 | 0 | 0 | 1 | 0 | 0 | 0 | 4.2 | 3.9 |
| 38 | 0 | 0 | 1 | 0 | 0 | 0 | 0 | 1 | 1 | 1 | 0 | 1 | 0 | 0 | 1 | 0 | 0 | 0 | 3.8 | 4.3 |
| 39 | 0 | 0 | 0 | 0 | 0 | 0 | 0 | 1 | 0 | 0 | 1 | 1 | 0 | 0 | 0 | 0 | 0 | 0 | 5.5 | 2.5 |
| 40 | 0 | 0 | 1 | 0 | 0 | 0 | 0 | 0 | 0 | 1 | 0 | 1 | 0 | 0 | 0 | 0 | 0 | 0 | 2.6 | 4.7 |
| 41 | 0 | 0 | 1 | 1 | 0 | 0 | 0 | 1 | 0 | 0 | 1 | 1 | 0 | 1 | 1 | 0 | 0 | 0 | 3.2 | 4.0 |
| 42 | 0 | 0 | 1 | 1 | 0 | 0 | 0 | 1 | 0 | 1 | 0 | 1 | 0 | 0 | 0 | 0 | 0 | 0 | 6.5 | 0.8 |
| 43 | 0 | 0 | 1 | 0 | 0 | 0 | 0 | 0 | 0 | 0 | 0 | 0 | 0 | 0 | 0 | 0 | 0 | 0 | 4.4 | 2.8 |
| 44 | 0 | 0 | 1 | 0 | 0 | 0 | 0 | 1 | 1 | 1 | 0 | 1 | 0 | 0 | 0 | 0 | 0 | 0 | 3.2 | 3.7 |
| 45 | 0 | 0 | 1 | 1 | 0 | 0 | 0 | 1 | 0 | 0 | 0 | 1 | 0 | 0 | 0 | 0 | 0 | 0 | 5.7 | 0.6 |
| 46 | 0 | 0 | 1 | 0 | 0 | 0 | 0 | 1 | 1 | 0 | 0 | 1 | 0 | 0 | 0 | 0 | 0 | 0 | 3.7 | 2.5 |
| 47 | 0 | 1 | 0 | 0 | 0 | 0 | 0 | 0 | 1 | 1 | 0 | 0 | 0 | 0 | 1 | 1 | 0 | 0 | 3.5 | 2.2 |
| 48 | 1 | 0 | 1 | 1 | 0 | 0 | 0 | 1 | 0 | 1 | 0 | 1 | 0 | 1 | 1 | 0 | 0 | 0 | 3.0 | 2.2 |
| 49 | 0 | 0 | 1 | 0 | 0 | 0 | 0 | 1 | 0 | 1 | 0 | 0 | 0 | 0 | 0 | 0 | 0 | 0 | 2.4 | 2.7 |
| 50 | 0 | 1 | 1 | 0 | 0 | 0 | 0 | 1 | 1 | 1 | 0 | 1 | 0 | 0 | 1 | 0 | 0 | 0 | 2.5 | 2.4 |
| 51 | 0 | 0 | 1 | 0 | 0 | 0 | 0 | 1 | 1 | 1 | 1 | 1 | 0 | 0 | 1 | 0 | 0 | 0 | 2.8 | 2.0 |
| 52 | 0 | 0 | 1 | 1 | 0 | 0 | 0 | 1 | 0 | 0 | 0 | 1 | 0 | 1 | 1 | 0 | 0 | 0 | 3.5 | 1.0 |
| 53 | 0 | 0 | 1 | 0 | 0 | 0 | 0 | 1 | 0 | 0 | 0 | 0 | 0 | 0 | 1 | 0 | 0 | 0 | 3.1 | 1.0 |
| 54 | 0 | 1 | 1 | 0 | 0 | 0 | 0 | 0 | 1 | 1 | 0 | 0 | 0 | 0 | 1 | 1 | 0 | 0 | 2.6 | 1.5 |
| 55 | 0 | 0 | 1 | 0 | 0 | 0 | 0 | 1 | 0 | 1 | 0 | 0 | 0 | 0 | 1 | 0 | 0 | 0 | 2.2 | 2.0 |
| 58 | 0 | 0 | 1 | 1 | 0 | 0 | 0 | 1 | 1 | 1 | 0 | 1 | 0 | 0 | 1 | 0 | 0 | 0 | 2.2 | 1.7 |
| 57 | 1 | 0 | 1 | 1 | 0 | 0 | 1 | 1 | 0 | 1 | 1 | 1 | 0 | 1 | 1 | 0 | 0 | 0 | 1.3 | 2.4 |
| 58 | 0 | 0 | 0 | 0 | 0 | 0 | 0 | 1 | 1 | 1 | 0 | 1 | 0 | 0 | 0 | 0 | 0 | 0 | 0.7 | 2.9 |
| 59 | 1 | 0 | 1 | 1 | 0 | 0 | 0 | 1 | 0 | 1 | 1 | 1 | 0 | 0 | 1 | 0 | 0 | 0 | 2.0 | 1.5 |
| 60 | 0 | 0 | 1 | 0 | 0 | 0 | 0 | 0 | 0 | 1 | 0 | 1 | 0 | 0 | 1 | 0 | 0 | 0 | 2.0 | 1.5 |
| 61 | 0 | 0 | 0 | 0 | 0 | 0 | 0 | 1 | 0 | 1 | 1 | 1 | 0 | 0 | 1 | 0 | 0 | 0 | 1.8 | 1.6 |
| 62 | 0 | 0 | 1 | 0 | 0 | 0 | 0 | 0 | 0 | 0 | 0 | 1 | 0 | 0 | 1 | 0 | 0 | 0 | 2.8 | 0.6 |
| 63 | 0 | 0 | 0 | 0 | 0 | 0 | 0 | 1 | 0 | 1 | 0 | 0 | 0 | 0 | 1 | 0 | 0 | 0 | 1.7 | 1.6 |
| 64 | 0 | 0 | 0 | 0 | 0 | 0 | 0 | 1 | 1 | 0 | 0 | 1 | 0 | 0 | 0 | 0 | 0 | 0 | 1.2 | 2.1 |
| 65 | 0 | 0 | 0 | 0 | 0 | 0 | 0 | 0 | 0 | 0 | 1 | 1 | 0 | 0 | 0 | 0 | 0 | 0 | 2.5 | 0.7 |
| 66 | 1 | 0 | 1 | 0 | 0 | 0 | 0 | 1 | 0 | 1 | 0 | 1 | 0 | 1 | 1 | 0 | 0 | 0 | 2.2 | 1.0 |
| 67 | 0 | 0 | 1 | 1 | 0 | 0 | 0 | 1 | 1 | 1 | 1 | 1 | 0 | 1 | 1 | 0 | 0 | 0 | 1.3 | 1.8 |
| 68 | 0 | 0 | 0 | 0 | 0 | 0 | 0 | 0 | 0 | 0 | 1 | 0 | 0 | 0 | 0 | 0 | 0 | 0 | 3.0 | 0.1 |
| 69 | 0 | 0 | 1 | 0 | 0 | 0 | 0 | 0 | 0 | 0 | 1 | 1 | 0 | 0 | 0 | 0 | 0 | 0 | 1.9 | 1.2 |
| 70 | 0 | 1 | 0 | 0 | 0 | 0 | 0 | 0 | 1 | 1 | 0 | 0 | 0 | 0 | 0 | 1 | 0 | 0 | 1.4 | 1.5 |
| 71 | 0 | 1 | 1 | 0 | 0 | 0 | 0 | 1 | 1 | 1 | 0 | 1 | 0 | 0 | 0 | 0 | 0 | 0 | 2.2 | 0.7 |
| 72 | 0 | 0 | 1 | 0 | 0 | 0 | 0 | 1 | 0 | 1 | 0 | 1 | 0 | 0 | 1 | 1 | 0 | 0 | 1.8 | 1.0 |
| 73 | 0 | 0 | 0 | 1 | 0 | 0 | 0 | 1 | 0 | 1 | 0 | 1 | 0 | 0 | 1 | 0 | 0 | 0 | 1.7 | 1.2 |
| 74 | 0 | 0 | 0 | 1 | 0 | 0 | 0 | 0 | 0 | 0 | 0 | 0 | 0 | 0 | 1 | 0 | 0 | 0 | 1.4 | 1.4 |
| 75 | 0 | 0 | 0 | 0 | 0 | 0 | 0 | 1 | 0 | 0 | 1 | 1 | 0 | 0 | 1 | 0 | 0 | 0 | 1.9 | 0.8 |
| 76 | 0 | 0 | 0 | 0 | 0 | 0 | 0 | 1 | 0 | 0 | 0 | 0 | 0 | 0 | 1 | 0 | 0 | 0 | 1.1 | 1.5 |
| 77 | 0 | 1 | 1 | 0 | 0 | 0 | 0 | 0 | 1 | 1 | 0 | 0 | 0 | 0 | 1 | 0 | 0 | 0 | 1.1 | 1.4 |
| 78 | 0 | 0 | 0 | 0 | 0 | 0 | 0 | 0 | 0 | 1 | 0 | 1 | 0 | 0 | 1 | 0 | 0 | 0 | 1.0 | 1.5 |
| 79 | 0 | 0 | 0 | 0 | 0 | 0 | 0 | 0 | 0 | 0 | 0 | 1 | 0 | 0 | 1 | 0 | 0 | 0 | 2.0 | 0.3 |
| 80 | 1 | 0 | 1 | 0 | 0 | 0 | 1 | 1 | 0 | 1 | 1 | 1 | 0 | 1 | 1 | 0 | 0 | 0 | 1.7 | 0.6 |
| 81 | 0 | 0 | 0 | 0 | 0 | 0 | 0 | 0 | 0 | 1 | 0 | 0 | 0 | 0 | 1 | 0 | 0 | 0 | 1.1 | 1.2 |
| 82 | 0 | 0 | 1 | 1 | 0 | 0 | 0 | 1 | 1 | 1 | 1 | 1 | 0 | 0 | 1 | 0 | 0 | 0 | 0.7 | 1.4 |
| 83 | 0 | 0 | 1 | 0 | 0 | 0 | 0 | 1 | 1 | 1 | 1 | 1 | 0 | 1 | 1 | 0 | 0 | 0 | 1.3 | 0.7 |
| 84 | 1 | 0 | 1 | 1 | 0 | 0 | 0 | 1 | 0 | 1 | 1 | 1 | 0 | 1 | 1 | 1 | 0 | 0 | 1.2 | 0.8 |
| 85 | 0 | 1 | 1 | 0 | 0 | 0 | 0 | 1 | 1 | 1 | 0 | 1 | 0 | 1 | 1 | 0 | 0 | 0 | 1.2 | 0.7 |
| 86 | 1 | 0 | 1 | 1 | 0 | 0 | 0 | 1 | 1 | 1 | 1 | 1 | 0 | 1 | 1 | 0 | 0 | 0 | 0.7 | 1.2 |
| 87 | 0 | 0 | 0 | 1 | 0 | 0 | 0 | 1 | 0 | 0 | 0 | 1 | 0 | 0 | 0 | 0 | 0 | 0 | 1.6 | 0.2 |
| 88 | 0 | 0 | 0 | 0 | 0 | 0 | 0 | 1 | 1 | 1 | 0 | 0 | 0 | 0 | 0 | 0 | 0 | 0 | 0.6 | 1.2 |
| 89 | 0 | 0 | 0 | 0 | 0 | 0 | 0 | 0 | 1 | 0 | 0 | 1 | 0 | 0 | 0 | 0 | 0 | 0 | 0.4 | 1.4 |
| 90 | 0 | 0 | 1 | 0 | 0 | 0 | 0 | 0 | 0 | 0 | 0 | 0 | 0 | 0 | 1 | 0 | 0 | 0 | 1.6 | 0.1 |
| 91 | 0 | 0 | 1 | 0 | 0 | 0 | 0 | 1 | 1 | 1 | 0 | 1 | 0 | 1 | 1 | 0 | 0 | 0 | 1.2 | 0.3 |
| 92 | 0 | 0 | 1 | 0 | 0 | 0 | 0 | 1 | 1 | 0 | 0 | 1 | 0 | 0 | 1 | 0 | 0 | 0 | 1.0 | 0.6 |
| 93 | 0 | 1 | 1 | 1 | 0 | 0 | 0 | 1 | 1 | 1 | 0 | 1 | 0 | 0 | 1 | 0 | 0 | 0 | 0.8 | 0.7 |
| 94 | 0 | 0 | 0 | 0 | 0 | 0 | 0 | 1 | 1 | 0 | 0 | 0 | 0 | 0 | 0 | 0 | 0 | 0 | 0.5 | 1.0 |
| 95 | 0 | 0 | 0 | 0 | 0 | 0 | 0 | 0 | 1 | 1 | 0 | 1 | 0 | 0 | 0 | 0 | 0 | 0 | 0.1 | 1.4 |
| 96 | 0 | 0 | 1 | 1 | 0 | 0 | 0 | 1 | 1 | 1 | 0 | 1 | 0 | 1 | 1 | 0 | 0 | 0 | 1.1 | 0.3 |
| 97 | 0 | 1 | 1 | 0 | 0 | 0 | 0 | 1 | 1 | 1 | 0 | 1 | 0 | 0 | 1 | 1 | 0 | 0 | 0.7 | 0.7 |
| 98 | 0 | 1 | 1 | 0 | 0 | 0 | 0 | 1 | 1 | 1 | 0 | 0 | 0 | 0 | 1 | 1 | 0 | 0 | 0.5 | 0.9 |
| 99 | 0 | 0 | 0 | 0 | 0 | 0 | 0 | 1 | 1 | 1 | 0 | 1 | 0 | 0 | 1 | 0 | 0 | 0 | 0.5 | 0.9 |
| 100 | 0 | 1 | 0 | 0 | 0 | 0 | 0 | 0 | 1 | 0 | 0 | 0 | 0 | 0 | 0 | 0 | 0 | 0 | 1.0 | 0.3 |
| 101 | 0 | 1 | 0 | 0 | 0 | 0 | 0 | 1 | 1 | 1 | 0 | 1 | 0 | 0 | 0 | 0 | 0 | 0 | 0.8 | 0.5 |
| 102 | 0 | 0 | 1 | 1 | 0 | 0 | 0 | 1 | 0 | 1 | 0 | 1 | 0 | 1 | 1 | 1 | 0 | 0 | 0.7 | 0.6 |
| 103 | 0 | 1 | 1 | 0 | 0 | 0 | 0 | 1 | 1 | 0 | 0 | 1 | 0 | 0 | 0 | 0 | 0 | 0 | 0.6 | 0.7 |
| 104 | 0 | 0 | 1 | 0 | 0 | 0 | 0 | 0 | 1 | 0 | 0 | 1 | 0 | 0 | 0 | 0 | 0 | 0 | 0.5 | 0.8 |
| 105 | 0 | 0 | 0 | 0 | 0 | 0 | 0 | 1 | 0 | 1 | 1 | 1 | 0 | 0 | 0 | 0 | 0 | 0 | 0.4 | 0.9 |
| 108 | 0 | 0 | 1 | 1 | 0 | 0 | 0 | 1 | 0 | 1 | 1 | 1 | 0 | 0 | 1 | 1 | 0 | 0 | 0.2 | 1.0 |
| 107 | 0 | 0 | 0 | 1 | 0 | 0 | 0 | 1 | 0 | 0 | 0 | 1 | 0 | 0 | 1 | 0 | 0 | 0 | 1.0 | 0.2 |
| 108 | 0 | 0 | 1 | 0 | 0 | 0 | 0 | 1 | 0 | 1 | 1 | 1 | 0 | 0 | 1 | 1 | 0 | 0 | 0.6 | 0.6 |
| 109 | 0 | 0 | 0 | 1 | 0 | 0 | 0 | 0 | 0 | 0 | 0 | 0 | 0 | 0 | 0 | 0 | 0 | 0 | 0.6 | 0.6 |
| 110 | 0 | 1 | 1 | 0 | 0 | 0 | 0 | 1 | 1 | 1 | 1 | 1 | 0 | 0 | 1 | 0 | 0 | 0 | 0.4 | 0.8 |
| 111 | 0 | 0 | 1 | 0 | 0 | 0 | 0 | 0 | 0 | 1 | 0 | 0 | 0 | 0 | 0 | 0 | 0 | 0 | 0.4 | 0.8 |
| 112 | 1 | 0 | 1 | 0 | 0 | 0 | 0 | 1 | 0 | 1 | 1 | 1 | 0 | 0 | 1 | 0 | 0 | 0 | 0.8 | 0.2 |
| 113 | 0 | 1 | 1 | 0 | 0 | 0 | 0 | 1 | 1 | 1 | 1 | 1 | 0 | 1 | 1 | 0 | 0 | 0 | 0.8 | 0.2 |
| 114 | 0 | 1 | 1 | 0 | 0 | 0 | 0 | 0 | 1 | 1 | 0 | 0 | 0 | 0 | 0 | 0 | 0 | 0 | 0.8 | 0.2 |
| 115 | 0 | 0 | 1 | 0 | 0 | 0 | 0 | 0 | 0 | 1 | 1 | 1 | 0 | 0 | 1 | 0 | 0 | 0 | 0.7 | 0.3 |
| 116 | 1 | 0 | 1 | 1 | 0 | 0 | 0 | 1 | 0 | 1 | 0 | 1 | 0 | 0 | 1 | 0 | 0 | 0 | 0.5 | 0.6 |
| 117 | 1 | 0 | 1 | 1 | 0 | 0 | 0 | 1 | 0 | 0 | 1 | 1 | 0 | 1 | 1 | 0 | 0 | 0 | 0.5 | 0.6 |
| 118 | 1 | 0 | 1 | 1 | 0 | 0 | 1 | 1 | 0 | 1 | 1 | 1 | 0 | 1 | 1 | 1 | 0 | 0 | 0.4 | 0.7 |
| 119 | 0 | 0 | 0 | 0 | 0 | 0 | 0 | 1 | 0 | 0 | 1 | 0 | 0 | 0 | 0 | 0 | 0 | 0 | 0.8 | 0.1 |
| 120 | 0 | 1 | 1 | 1 | 0 | 0 | 0 | 1 | 1 | 1 | 1 | 1 | 0 | 1 | 1 | 0 | 0 | 0 | 0.7 | 0.2 |
| 121 | 0 | 0 | 1 | 0 | 0 | 0 | 0 | 1 | 1 | 0 | 1 | 1 | 0 | 0 | 1 | 0 | 0 | 0 | 0.7 | 0.2 |
| 122 | 0 | 1 | 1 | 0 | 0 | 0 | 0 | 0 | 1 | 1 | 0 | 0 | 0 | 0 | 0 | 1 | 0 | 0 | 0.6 | 0.3 |
| 123 | 0 | 0 | 0 | 0 | 0 | 0 | 0 | 0 | 1 | 1 | 0 | 0 | 0 | 0 | 1 | 0 | 0 | 0 | 0.6 | 0.3 |
| 124 | 1 | 1 | 0 | 0 | 0 | 0 | 0 | 0 | 1 | 1 | 0 | 0 | 0 | 0 | 1 | 1 | 0 | 0 | 0.5 | 0.5 |
| 125 | 0 | 1 | 0 | 0 | 0 | 0 | 0 | 1 | 1 | 1 | 0 | 0 | 0 | 0 | 1 | 0 | 0 | 0 | 0.5 | 0.5 |
| 126 | 0 | 0 | 1 | 0 | 0 | 0 | 1 | 1 | 0 | 1 | 1 | 1 | 0 | 1 | 1 | 0 | 0 | 0 | 0.5 | 0.5 |
| 127 | 0 | 0 | 1 | 0 | 0 | 0 | 0 | 1 | 1 | 1 | 1 | 1 | 0 | 0 | 0 | 0 | 0 | 0 | 0.5 | 0.5 |
| 128 | 1 | 1 | 1 | 0 | 0 | 0 | 0 | 0 | 1 | 1 | 0 | 0 | 0 | 0 | 1 | 1 | 0 | 0 | 0.2 | 0.7 |
| 129 | 0 | 1 | 1 | 0 | 0 | 0 | 0 | 1 | 1 | 1 | 0 | 0 | 0 | 0 | 1 | 0 | 0 | 0 | 0.2 | 0.7 |
| 130 | 1 | 0 | 1 | 1 | 0 | 0 | 0 | 1 | 0 | 1 | 0 | 1 | 0 | 1 | 1 | 1 | 0 | 0 | 0.6 | 0.2 |
| 131 | 1 | 0 | 1 | 0 | 0 | 0 | 0 | 1 | 0 | 1 | 0 | 1 | 0 | 0 | 1 | 0 | 0 | 0 | 0.5 | 0.3 |
| 133 | 0 | 1 | 1 | 1 | 0 | 0 | 0 | 1 | 1 | 1 | 0 | 1 | 0 | 0 | 1 | 1 | 0 | 0 | 0.5 | 0.3 |
| 133 | 0 | 0 | 1 | 0 | 0 | 0 | 0 | 0 | 1 | 1 | 0 | 1 | 0 | 0 | 0 | 0 | 0 | 0 | 0.5 | 0.3 |
| 134 | 0 | 1 | 0 | 0 | 0 | 0 | 0 | 1 | 1 | 1 | 0 | 0 | 0 | 0 | 1 | 1 | 0 | 0 | 0.4 | 0.5 |
| 135 | 0 | 1 | 0 | 0 | 0 | 0 | 0 | 1 | 1 | 1 | 0 | 0 | 0 | 0 | 0 | 0 | 0 | 0 | 0.4 | 0.5 |
| 136 | 0 | 0 | 1 | 1 | 0 | 0 | 0 | 1 | 0 | 1 | 0 | 1 | 0 | 0 | 1 | 1 | 0 | 0 | 0.4 | 0.5 |
| 137 | 0 | 0 | 1 | 1 | 0 | 0 | 0 | 0 | 0 | 0 | 0 | 0 | 0 | 0 | 1 | 0 | 0 | 0 | 0.4 | 0.5 |
| 138 | 0 | 0 | 0 | 0 | 0 | 0 | 0 | 0 | 1 | 0 | 0 | 1 | 0 | 0 | 1 | 0 | 0 | 0 | 0.2 | 0.6 |
| 139 | 0 | 1 | 1 | 0 | 0 | 0 | 0 | 0 | 1 | 1 | 0 | 1 | 0 | 0 | 0 | 0 | 0 | 0 | 0.6 | 0.1 |
| 140 | 0 | 0 | 0 | 0 | 0 | 0 | 0 | 0 | 0 | 0 | 1 | 1 | 0 | 0 | 1 | 0 | 0 | 0 | 0.6 | 0.1 |
| 141 | 1 | 0 | 1 | 0 | 0 | 0 | 0 | 1 | 0 | 1 | 1 | 1 | 0 | 1 | 1 | 1 | 0 | 0 | 0.5 | 0.2 |
| 142 | 0 | 0 | 1 | 1 | 0 | 0 | 0 | 0 | 0 | 1 | 0 | 1 | 0 | 0 | 1 | 0 | 0 | 0 | 0.5 | 0.2 |
| 143 | 0 | 1 | 1 | 1 | 0 | 0 | 0 | 1 | 1 | 1 | 1 | 1 | 0 | 0 | 1 | 0 | 0 | 0 | 0.4 | 0.3 |
| 144 | 0 | 0 | 1 | 1 | 0 | 0 | 0 | 1 | 0 | 1 | 1 | 1 | 0 | 0 | 0 | 0 | 0 | 0 | 0.4 | 0.3 |
| 145 | 0 | 0 | 1 | 0 | 0 | 0 | 0 | 0 | 0 | 1 | 0 | 0 | 0 | 0 | 1 | 0 | 0 | 0 | 0.4 | 0.3 |
| 146 | 0 | 1 | 1 | 0 | 0 | 0 | 0 | 1 | 1 | 0 | 0 | 1 | 0 | 0 | 1 | 0 | 0 | 0 | 0.2 | 0.5 |
| 147 | 1 | 1 | 1 | 1 | 0 | 0 | 0 | 1 | 1 | 1 | 0 | 0 | 0 | 1 | 1 | 1 | 0 | 0 | 0.5 | 0.1 |
| 148 | 1 | 1 | 0 | 0 | 0 | 0 | 0 | 0 | 1 | 1 | 0 | 0 | 0 | 1 | 1 | 1 | 0 | 0 | 0.5 | 0.1 |
| 149 | 0 | 0 | 0 | 1 | 0 | 0 | 0 | 1 | 0 | 1 | 1 | 1 | 0 | 0 | 1 | 0 | 0 | 0 | 0.5 | 0.1 |
| 150 | 0 | 0 | 0 | 0 | 0 | 0 | 0 | 1 | 0 | 1 | 0 | 1 | 0 | 1 | 1 | 0 | 0 | 0 | 0.5 | 0.1 |
| 151 | 0 | 1 | 1 | 0 | 0 | 0 | 0 | 0 | 1 | 1 | 0 | 1 | 0 | 0 | 1 | 0 | 0 | 0 | 0.4 | 0.2 |
| 152 | 0 | 1 | 0 | 0 | 0 | 0 | 0 | 1 | 1 | 1 | 0 | 1 | 0 | 0 | 1 | 0 | 0 | 0 | 0.4 | 0.2 |
| 153 | 0 | 0 | 1 | 1 | 0 | 0 | 0 | 1 | 0 | 1 | 1 | 1 | 0 | 1 | 1 | 1 | 0 | 0 | 0.4 | 0.2 |
| 154 | 0 | 0 | 0 | 0 | 0 | 0 | 0 | 0 | 1 | 0 | 0 | 0 | 0 | 0 | 1 | 0 | 0 | 0 | 0.4 | 0.2 |
| 155 | 1 | 1 | 1 | 0 | 0 | 0 | 0 | 1 | 1 | 1 | 0 | 0 | 0 | 1 | 1 | 1 | 0 | 0 | 0.2 | 0.3 |
| 156 | 0 | 1 | 0 | 0 | 0 | 0 | 0 | 0 | 1 | 1 | 0 | 1 | 0 | 0 | 0 | 0 | 0 | 0 | 0.2 | 0.3 |
| 157 | 0 | 0 | 1 | 0 | 0 | 0 | 0 | 1 | 0 | 0 | 0 | 1 | 0 | 0 | 0 | 1 | 0 | 0 | 0.2 | 0.3 |
| 158 | 1 | 0 | 0 | 1 | 0 | 0 | 0 | 0 | 0 | 0 | 0 | 0 | 0 | 0 | 1 | 0 | 0 | 0 | 0.1 | 0.5 |
| 159 | 0 | 1 | 0 | 0 | 0 | 0 | 0 | 1 | 1 | 0 | 0 | 1 | 0 | 0 | 0 | 0 | 0 | 0 | 0.1 | 0.5 |
| 160 | 0 | 0 | 1 | 0 | 0 | 0 | 0 | 1 | 0 | 1 | 0 | 1 | 0 | 1 | 1 | 1 | 0 | 0 | 0.1 | 0.5 |
| 161 | 0 | 0 | 0 | 0 | 0 | 0 | 0 | 1 | 1 | 0 | 0 | 1 | 0 | 0 | 1 | 0 | 0 | 0 | 0.1 | 0.5 |
| 162 | 0 | 1 | 1 | 1 | 0 | 0 | 0 | 1 | 1 | 1 | 0 | 1 | 0 | 1 | 1 | 0 | 0 | 0 | 0.4 | 0.1 |
| 163 | 0 | 1 | 1 | 0 | 0 | 0 | 0 | 1 | 1 | 1 | 0 | 0 | 0 | 1 | 1 | 1 | 0 | 0 | 0.4 | 0.1 |
| 164 | 0 | 1 | 0 | 0 | 0 | 0 | 0 | 0 | 1 | 1 | 0 | 1 | 0 | 0 | 1 | 0 | 0 | 0 | 0.4 | 0.1 |
| 165 | 0 | 0 | 1 | 0 | 0 | 0 | 0 | 1 | 0 | 0 | 0 | 1 | 0 | 0 | 1 | 1 | 0 | 0 | 0.4 | 0.1 |
| 166 | 1 | 1 | 1 | 1 | 0 | 0 | 1 | 1 | 1 | 1 | 1 | 1 | 0 | 1 | 1 | 1 | 0 | 0 | 0.2 | 0.2 |
| 167 | 1 | 1 | 1 | 1 | 0 | 0 | 0 | 0 | 1 | 1 | 0 | 0 | 0 | 0 | 1 | 1 | 0 | 0 | 0.2 | 0.2 |
| 168 | 0 | 1 | 1 | 0 | 0 | 0 | 0 | 1 | 1 | 1 | 0 | 0 | 0 | 0 | 0 | 0 | 0 | 0 | 0.2 | 0.2 |
| 169 | 0 | 0 | 1 | 1 | 0 | 0 | 0 | 1 | 0 | 0 | 0 | 0 | 0 | 0 | 1 | 0 | 0 | 0 | 0.2 | 0.2 |
| 170 | 0 | 0 | 1 | 1 | 0 | 0 | 0 | 0 | 0 | 0 | 0 | 1 | 0 | 0 | 1 | 0 | 0 | 0 | 0.2 | 0.2 |
| 171 | 0 | 0 | 1 | 0 | 0 | 0 | 0 | 1 | 1 | 0 | 0 | 0 | 0 | 0 | 0 | 0 | 0 | 0 | 0.2 | 0.2 |
| 172 | 0 | 0 | 0 | 0 | 0 | 0 | 0 | 1 | 0 | 0 | 0 | 1 | 0 | 1 | 1 | 0 | 0 | 0 | 0.2 | 0.2 |
| 173 | 0 | 1 | 1 | 1 | 0 | 0 | 0 | 1 | 1 | 1 | 0 | 0 | 0 | 1 | 1 | 1 | 0 | 0 | 0.1 | 0.3 |
| 174 | 1 | 1 | 1 | 1 | 0 | 0 | 0 | 0 | 1 | 1 | 0 | 0 | 0 | 1 | 1 | 1 | 0 | 0 | 0.2 | 0.1 |
| 175 | 1 | 0 | 1 | 1 | 0 | 0 | 0 | 1 | 1 | 1 | 0 | 1 | 0 | 1 | 1 | 0 | 0 | 0 | 0.2 | 0.1 |
| 176 | 1 | 0 | 1 | 0 | 0 | 0 | 0 | 1 | 0 | 0 | 1 | 1 | 0 | 0 | 1 | 0 | 0 | 0 | 0.2 | 0.1 |
| 177 | 1 | 0 | 0 | 0 | 0 | 0 | 0 | 0 | 0 | 0 | 0 | 0 | 0 | 0 | 1 | 0 | 0 | 0 | 0.2 | 0.1 |
| 178 | 0 | 0 | 0 | 1 | 0 | 0 | 0 | 1 | 0 | 1 | 0 | 1 | 0 | 1 | 1 | 0 | 0 | 0 | 0.2 | 0.1 |
| 179 | 0 | 0 | 0 | 0 | 0 | 0 | 0 | 0 | 1 | 1 | 0 | 1 | 0 | 0 | 1 | 0 | 0 | 0 | 0.2 | 0.1 |
| 180 | 1 | 1 | 1 | 1 | 0 | 0 | 0 | 1 | 1 | 1 | 1 | 1 | 0 | 1 | 1 | 0 | 0 | 0 | 0.1 | 0.2 |
| 181 | 1 | 0 | 1 | 1 | 0 | 0 | 1 | 1 | 0 | 1 | 0 | 1 | 0 | 1 | 1 | 0 | 0 | 0 | 0.1 | 0.2 |
| 162 | 0 | 1 | 0 | 0 | 0 | 0 | 0 | 0 | 1 | 1 | 0 | 0 | 0 | 1 | 1 | 0 | 0 | 0 | 0.1 | 0.2 |
| 183 | 0 | 0 | 1 | 1 | 0 | 0 | 0 | 1 | 0 | 1 | 0 | 0 | 0 | 0 | 1 | 0 | 0 | 0 | 0.1 | 0.2 |
| 184 | 0 | 0 | 1 | 0 | 0 | 0 | 0 | 1 | 1 | 0 | 1 | 1 | 0 | 1 | 1 | 0 | 0 | 0 | 0.1 | 0.2 |
| 185 | 0 | 0 | 1 | 0 | 0 | 0 | 0 | 1 | 1 | 0 | 1 | 1 | 0 | 0 | 0 | 0 | 0 | 0 | 0.1 | 0.2 |
| 186 | 0 | 0 | 0 | 0 | 0 | 0 | 0 | 1 | 1 | 0 | 1 | 1 | 0 | 0 | 0 | 0 | 0 | 0 | 0.1 | 0.2 |
| 187 | 1 | 1 | 1 | 0 | 0 | 0 | 0 | 0 | 1 | 1 | 0 | 0 | 0 | 0 | 1 | 0 | 0 | 0 | 0.1 | 0.1 |
| 188 | 1 | 0 | 1 | 0 | 0 | 0 | 1 | 1 | 0 | 1 | 1 | 1 | 0 | 1 | 1 | 1 | 0 | 0 | 0.1 | 0.1 |
| 189 | 0 | 1 | 1 | 1 | 0 | 0 | 0 | 1 | 1 | 1 | 0 | 0 | 0 | 0 | 1 | 1 | 0 | 0 | 0.1 | 0.1 |
| 190 | 0 | 1 | 1 | 1 | 0 | 0 | 0 | 1 | 1 | 0 | 0 | 1 | 0 | 0 | 1 | 0 | 0 | 0 | 0.1 | 0.1 |
| 191 | 0 | 1 | 0 | 0 | 0 | 0 | 0 | 0 | 1 | 1 | 1 | 1 | 0 | 0 | 1 | 0 | 0 | 0 | 0.1 | 0.1 |
| 192 | 0 | 0 | 1 | 1 | 0 | 0 | 0 | 1 | 1 | 1 | 1 | 1 | 0 | 1 | 1 | 1 | 0 | 0 | 0.1 | 0.1 |
| 193 | 0 | 0 | 1 | 1 | 0 | 0 | 0 | 1 | 0 | 0 | 1 | 1 | 0 | 0 | 0 | 0 | 0 | 0 | 0.1 | 0.1 |
| 194 | 0 | 0 | 1 | 0 | 0 | 0 | 1 | 1 | 0 | 0 | 1 | 1 | 0 | 1 | 1 | 0 | 0 | 0 | 0.1 | 0.1 |
| 195 | 0 | 0 | 1 | 0 | 0 | 0 | 0 | 1 | 0 | 0 | 1 | 1 | 0 | 0 | 1 | 1 | 0 | 0 | 0.1 | 0.1 |
| 198 | 0 | 0 | 1 | 0 | 0 | 0 | 0 | 1 | 0 | 0 | 1 | 0 | 0 | 0 | 0 | 0 | 0 | 0 | 0.1 | 0.1 |
| 197 | 0 | 0 | 1 | 0 | 0 | 0 | 0 | 1 | 0 | 0 | 0 | 0 | 0 | 1 | 1 | 0 | 0 | 0 | 0.1 | 0.1 |
| 198 | 0 | 0 | 0 | 1 | 0 | 0 | 0 | 0 | 0 | 1 | 1 | 1 | 0 | 0 | 1 | 0 | 0 | 0 | 0.1 | 0.1 |
| 199 | 0 | 0 | 0 | 1 | 0 | 0 | 0 | 0 | 0 | 1 | 0 | 0 | 0 | 0 | 1 | 0 | 0 | 0 | 0.1 | 0.1 |
| 200 | 0 | 0 | 0 | 1 | 0 | 0 | 0 | 0 | 0 | 0 | 0 | 1 | 0 | 0 | 1 | 0 | 0 | 0 | 0.1 | 0.1 |
| 201 | 0 | 0 | 0 | 1 | 0 | 0 | 0 | 0 | 0 | 0 | 0 | 1 | 0 | 0 | 0 | 0 | 0 | 0 | 0.1 | 0.1 |
| 202 | 0 | 0 | 0 | 0 | 0 | 0 | 0 | 1 | 0 | 0 | 1 | 1 | 0 | 1 | 1 | 0 | 0 | 0 | 0.1 | 0.1 |
| 203 | 0 | 0 | 0 | 0 | 0 | 0 | 0 | 1 | 0 | 0 | 0 | 1 | 0 | 0 | 0 | 1 | 0 | 0 | 0.1 | 0.1 |

| | | |
|---|---|---|
| total | 971.3 | 990.9 |

¹Protein code = binary code per line

TABLE 3

| | | | | | | | | | | | | | | | | | | | V1 | V2 |
|---|---|---|---|---|---|---|---|---|---|---|---|---|---|---|---|---|---|---|---|---|
| | | | | | | | | | | | | | | | | | | | No. of | No. of |
| | | | | | | | | | | | | | | | | | | | cells | cells |
| Protein code² | Proteins [1-18], binary | | | | | | | | | | | | | | | | | | In | In |
| No. | 1 | 2 | 3 | 4 | 5 | 6 | 7 | 8 | 9 | 10 | 11 | 12 | 13 | 14 | 15 | 16 | 17 | 18 | 1/1000 | 1/1000 |
| 1 | 0 | 0 | 1 | 0 | 0 | 0 | 0 | 0 | 0 | 0 | 1 | 1 | 0 | 0 | 1 | 0 | 0 | 0 | 1.7 | 0.0 |
| 2 | 0 | 0 | 0 | 1 | 0 | 0 | 0 | 1 | 0 | 1 | 0 | 1 | 0 | 0 | 0 | 0 | 0 | 0 | 1.2 | 0.0 |
| 3 | 0 | 1 | 1 | 1 | 0 | 0 | 0 | 1 | 1 | 1 | 0 | 1 | 0 | 1 | 1 | 1 | 0 | 0 | 0.8 | 0.0 |
| 4 | 1 | 0 | 1 | 0 | 0 | 0 | 0 | 1 | 0 | 0 | 1 | 1 | 0 | 1 | 1 | 0 | 0 | 0 | 0.7 | 0.0 |
| 5 | 0 | 0 | 1 | 0 | 0 | 0 | 1 | 1 | 0 | 1 | 0 | 1 | 0 | 1 | 1 | 0 | 0 | 0 | 0.7 | 0.0 |
| 6 | 0 | 0 | 0 | 1 | 0 | 0 | 0 | 0 | 0 | 0 | 0 | 0 | 0 | 0 | 1 | 1 | 0 | 0 | 0.7 | 0.0 |
| 7 | 1 | 0 | 1 | 0 | 0 | 0 | 0 | 1 | 1 | 1 | 1 | 1 | 0 | 1 | 1 | 0 | 0 | 0 | 0.6 | 0.0 |
| 8 | 0 | 0 | 1 | 0 | 0 | 0 | 0 | 0 | 0 | 0 | 1 | 0 | 0 | 0 | 0 | 0 | 0 | 0 | 0.6 | 0.0 |
| 9 | 1 | 1 | 1 | 1 | 0 | 0 | 0 | 1 | 1 | 1 | 0 | 1 | 0 | 1 | 1 | 1 | 0 | 0 | 0.5 | 0.0 |
| 10 | 0 | 0 | 1 | 1 | 0 | 0 | 0 | 1 | 1 | 1 | 0 | 1 | 0 | 0 | 0 | 0 | 0 | 0 | 0.5 | 0.0 |
| 11 | 0 | 0 | 0 | 0 | 0 | 0 | 0 | 1 | 1 | 1 | 1 | 1 | 0 | 0 | 1 | 0 | 0 | 0 | 0.5 | 0.0 |
| 12 | 1 | 1 | 1 | 0 | 0 | 0 | 0 | 1 | 1 | 1 | 0 | 1 | 0 | 1 | 1 | 1 | 0 | 0 | 0.4 | 0.0 |
| 13 | 1 | 1 | 0 | 0 | 0 | 0 | 0 | 0 | 1 | 1 | 0 | 0 | 0 | 0 | 1 | 0 | 0 | 0 | 0.4 | 0.0 |
| 14 | 1 | 0 | 1 | 1 | 0 | 0 | 0 | 1 | 1 | 1 | 1 | 1 | 0 | 0 | 1 | 0 | 0 | 0 | 0.4 | 0.0 |
| 15 | 1 | 0 | 1 | 0 | 0 | 0 | 0 | 1 | 0 | 0 | 0 | 1 | 0 | 1 | 1 | 0 | 0 | 0 | 0.4 | 0.0 |
| 16 | 1 | 0 | 0 | 1 | 0 | 0 | 0 | 0 | 0 | 1 | 0 | 0 | 0 | 1 | 1 | 0 | 0 | 0 | 0.4 | 0.0 |
| 17 | 1 | 0 | 0 | 1 | 0 | 0 | 0 | 0 | 0 | 0 | 0 | 1 | 0 | 0 | 1 | 1 | 0 | 0 | 0.4 | 0.0 |
| 18 | 1 | 0 | 0 | 0 | 0 | 0 | 0 | 0 | 0 | 0 | 0 | 0 | 0 | 1 | 1 | 0 | 0 | 0 | 0.4 | 0.0 |
| 19 | 0 | 1 | 1 | 0 | 0 | 0 | 0 | 1 | 1 | 1 | 0 | 1 | 0 | 1 | 1 | 1 | 0 | 0 | 0.4 | 0.0 |
| 20 | 0 | 1 | 1 | 0 | 0 | 0 | 0 | 1 | 1 | 0 | 0 | 0 | 0 | 0 | 0 | 0 | 0 | 0 | 0.4 | 0.0 |
| 21 | 0 | 1 | 0 | 0 | 0 | 0 | 0 | 0 | 1 | 1 | 0 | 1 | 0 | 0 | 1 | 1 | 0 | 0 | 0.4 | 0.0 |
| 22 | 0 | 1 | 0 | 0 | 0 | 0 | 0 | 0 | 1 | 1 | 0 | 0 | 0 | 1 | 1 | 1 | 0 | 0 | 0.4 | 0.0 |
| 23 | 0 | 0 | 1 | 1 | 0 | 0 | 0 | 1 | 1 | 0 | 1 | 1 | 0 | 0 | 1 | 0 | 0 | 0 | 0.4 | 0.0 |
| 24 | 0 | 0 | 1 | 1 | 0 | 0 | 0 | 1 | 1 | 0 | 0 | 1 | 0 | 0 | 1 | 0 | 0 | 0 | 0.4 | 0.0 |
| 25 | 0 | 0 | 1 | 0 | 0 | 0 | 0 | 1 | 0 | 1 | 0 | 1 | 1 | 0 | 1 | 0 | 0 | 0 | 0.4 | 0.0 |
| 26 | 0 | 0 | 1 | 0 | 0 | 0 | 0 | 1 | 0 | 0 | 1 | 0 | 0 | 0 | 1 | 0 | 0 | 0 | 0.4 | 0.0 |
| 27 | 0 | 0 | 0 | 0 | 0 | 0 | 0 | 0 | 0 | 0 | 1 | 0 | 0 | 0 | 1 | 0 | 0 | 0 | 0.4 | 0.0 |
| 28 | 1 | 1 | 1 | 0 | 0 | 0 | 0 | 1 | 1 | 1 | 1 | 1 | 0 | 1 | 1 | 1 | 0 | 0 | 0.2 | 0.0 |
| 29 | 1 | 1 | 0 | 0 | 0 | 0 | 0 | 1 | 1 | 1 | 0 | 0 | 0 | 1 | 1 | 1 | 0 | 0 | 0.2 | 0.0 |
| 30 | 1 | 0 | 1 | 1 | 0 | 0 | 0 | 0 | 0 | 0 | 0 | 0 | 0 | 0 | 1 | 1 | 0 | 0 | 0.2 | 0.0 |
| 31 | 1 | 0 | 1 | 0 | 0 | 0 | 1 | 1 | 1 | 1 | 1 | 1 | 0 | 1 | 1 | 0 | 0 | 0 | 0.2 | 0.0 |
| 32 | 0 | 1 | 1 | 1 | 0 | 0 | 0 | 1 | 1 | 1 | 1 | 1 | 0 | 1 | 1 | 1 | 0 | 0 | 0.2 | 0.0 |
| 33 | 0 | 1 | 1 | 1 | 0 | 0 | 0 | 1 | 1 | 1 | 0 | 0 | 0 | 0 | 1 | 0 | 0 | 0 | 0.2 | 0.0 |
| 34 | 0 | 1 | 1 | 1 | 0 | 0 | 0 | 0 | 1 | 1 | 0 | 0 | 0 | 0 | 1 | 1 | 0 | 0 | 0.2 | 0.0 |
| 35 | 0 | 0 | 1 | 1 | 0 | 0 | 0 | 0 | 0 | 0 | 1 | 1 | 0 | 0 | 1 | 0 | 0 | 0 | 0.2 | 0.0 |
| 36 | 0 | 0 | 1 | 0 | 0 | 0 | 0 | 1 | 1 | 1 | 0 | 0 | 0 | 0 | 0 | 0 | 0 | 0 | 0.2 | 0.0 |
| 37 | 0 | 0 | 1 | 0 | 0 | 0 | 0 | 1 | 0 | 1 | 1 | 1 | 0 | 1 | 1 | 1 | 0 | 0 | 0.2 | 0.0 |
| 38 | 0 | 0 | 1 | 0 | 0 | 0 | 0 | 1 | 0 | 1 | 1 | 0 | 0 | 0 | 1 | 0 | 0 | 0 | 0.2 | 0.0 |
| 39 | 0 | 0 | 1 | 0 | 0 | 0 | 0 | 0 | 1 | 0 | 1 | 1 | 0 | 0 | 0 | 0 | 0 | 0 | 0.2 | 0.0 |
| 40 | 0 | 0 | 0 | 1 | 0 | 0 | 0 | 1 | 0 | 1 | 0 | 0 | 0 | 0 | 1 | 0 | 0 | 0 | 0.2 | 0.0 |
| 41 | 0 | 0 | 0 | 0 | 0 | 0 | 0 | 1 | 1 | 0 | 1 | 1 | 0 | 0 | 1 | 0 | 0 | 0 | 0.2 | 0.0 |
| 42 | 0 | 0 | 0 | 0 | 0 | 0 | 0 | 1 | 0 | 1 | 1 | 0 | 0 | 0 | 1 | 0 | 0 | 0 | 0.2 | 0.0 |
| 43 | 0 | 0 | 0 | 0 | 0 | 0 | 0 | 1 | 0 | 0 | 1 | 0 | 0 | 0 | 1 | 0 | 0 | 0 | 0.2 | 0.0 |
| 44 | 0 | 0 | 0 | 0 | 0 | 0 | 0 | 0 | 0 | 0 | 0 | 0 | 0 | 0 | 1 | 1 | 0 | 0 | 0.2 | 0.0 |
| 45 | 1 | 1 | 1 | 1 | 0 | 0 | 1 | 0 | 1 | 1 | 0 | 0 | 0 | 1 | 1 | 0 | 0 | 0 | 0.1 | 0.0 |
| 46 | 1 | 1 | 1 | 1 | 0 | 0 | 0 | 1 | 1 | 1 | 0 | 1 | 0 | 0 | 1 | 1 | 0 | 0 | 0.1 | 0.0 |
| 47 | 1 | 1 | 1 | 1 | 0 | 0 | 0 | 1 | 1 | 1 | 0 | 0 | 1 | 1 | 1 | 1 | 0 | 0 | 0.1 | 0.0 |
| 48 | 1 | 1 | 1 | 1 | 0 | 0 | 0 | 1 | 1 | 1 | 0 | 0 | 0 | 1 | 1 | 0 | 0 | 0 | 0.1 | 0.0 |
| 49 | 1 | 1 | 1 | 0 | 0 | 0 | 0 | 1 | 1 | 1 | 1 | 1 | 0 | 1 | 1 | 0 | 0 | 0 | 0.1 | 0.0 |
| 50 | 1 | 1 | 1 | 0 | 0 | 0 | 0 | 1 | 1 | 1 | 0 | 1 | 0 | 1 | 1 | 0 | 0 | 0 | 0.1 | 0.0 |
| 51 | 1 | 1 | 1 | 0 | 0 | 0 | 0 | 0 | 1 | 1 | 0 | 0 | 0 | 1 | 1 | 1 | 0 | 0 | 0.1 | 0.0 |
| 52 | 1 | 1 | 0 | 0 | 0 | 0 | 0 | 0 | 1 | 1 | 0 | 1 | 0 | 1 | 1 | 1 | 0 | 0 | 0.1 | 0.0 |
| 53 | 1 | 1 | 0 | 0 | 0 | 0 | 0 | 0 | 1 | 1 | 0 | 0 | 0 | 0 | 0 | 1 | 0 | 0 | 0.1 | 0.0 |
| 54 | 1 | 0 | 1 | 1 | 0 | 0 | 0 | 1 | 1 | 0 | 1 | 1 | 0 | 1 | 1 | 0 | 0 | 0 | 0.1 | 0.0 |
| 55 | 1 | 0 | 1 | 1 | 0 | 0 | 0 | 1 | 0 | 1 | 1 | 1 | 0 | 0 | 1 | 1 | 0 | 0 | 0.1 | 0.0 |
| 56 | 1 | 0 | 1 | 1 | 0 | 0 | 0 | 1 | 0 | 0 | 1 | 1 | 0 | 0 | 1 | 0 | 0 | 0 | 0.1 | 0.0 |
| 57 | 1 | 0 | 1 | 1 | 0 | 0 | 0 | 0 | 0 | 0 | 0 | 0 | 0 | 0 | 1 | 0 | 0 | 0 | 0.1 | 0.0 |
| 58 | 1 | 0 | 1 | 0 | 0 | 0 | 1 | 1 | 0 | 1 | 0 | 1 | 0 | 1 | 1 | 0 | 0 | 0 | 0.1 | 0.0 |
| 59 | 1 | 0 | 1 | 0 | 0 | 0 | 0 | 1 | 1 | 1 | 1 | 1 | 0 | 1 | 1 | 1 | 0 | 0 | 0.1 | 0.0 |
| 60 | 1 | 0 | 1 | 0 | 0 | 0 | 0 | 1 | 1 | 1 | 0 | 1 | 0 | 1 | 1 | 0 | 0 | 0 | 0.1 | 0.0 |
| 61 | 1 | 0 | 1 | 0 | 0 | 0 | 0 | 1 | 0 | 1 | 1 | 1 | 1 | 1 | 1 | 0 | 0 | 0 | 0.1 | 0.0 |
| 62 | 1 | 0 | 1 | 0 | 0 | 0 | 0 | 1 | 0 | 1 | 0 | 1 | 0 | 1 | 1 | 1 | 0 | 0 | 0.1 | 0.0 |
| 63 | 1 | 0 | 0 | 1 | 0 | 0 | 1 | 1 | 0 | 1 | 0 | 0 | 0 | 1 | 1 | 1 | 0 | 0 | 0.1 | 0.0 |
| 64 | 1 | 0 | 0 | 1 | 0 | 0 | 0 | 1 | 0 | 1 | 1 | 1 | 0 | 1 | 1 | 0 | 0 | 0 | 0.1 | 0.0 |
| 65 | 1 | 0 | 0 | 1 | 0 | 0 | 0 | 1 | 0 | 1 | 0 | 1 | 0 | 1 | 1 | 0 | 0 | 0 | 0.1 | 0.0 |
| 66 | 1 | 0 | 0 | 1 | 0 | 0 | 0 | 0 | 0 | 0 | 0 | 0 | 0 | 0 | 1 | 1 | 0 | 0 | 0.1 | 0.0 |
| 67 | 1 | 0 | 0 | 0 | 0 | 0 | 0 | 1 | 1 | 1 | 0 | 1 | 0 | 1 | 1 | 0 | 0 | 0 | 0.1 | 0.0 |
| 68 | 1 | 0 | 0 | 0 | 0 | 0 | 0 | 1 | 0 | 1 | 1 | 1 | 0 | 0 | 1 | 0 | 0 | 0 | 0.1 | 0.0 |
| 69 | 1 | 0 | 0 | 0 | 0 | 0 | 0 | 1 | 0 | 0 | 1 | 1 | 0 | 1 | 1 | 0 | 0 | 0 | 0.1 | 0.0 |
| 70 | 1 | 0 | 0 | 0 | 0 | 0 | 0 | 0 | 0 | 0 | 0 | 1 | 0 | 1 | 1 | 0 | 0 | 0 | 0.1 | 0.0 |
| 71 | 1 | 0 | 0 | 0 | 0 | 0 | 0 | 0 | 0 | 0 | 0 | 0 | 0 | 1 | 1 | 1 | 0 | 0 | 0.1 | 0.0 |
| 72 | 1 | 0 | 0 | 0 | 0 | 0 | 0 | 0 | 0 | 0 | 0 | 0 | 0 | 0 | 1 | 1 | 0 | 0 | 0.1 | 0.0 |
| 73 | 0 | 1 | 1 | 1 | 0 | 0 | 0 | 1 | 1 | 1 | 0 | 1 | 0 | 0 | 0 | 0 | 0 | 0 | 0.1 | 0.0 |
| 74 | 0 | 1 | 1 | 1 | 0 | 0 | 0 | 0 | 1 | 1 | 0 | 1 | 0 | 1 | 1 | 0 | 0 | 0 | 0.1 | 0.0 |
| 75 | 0 | 1 | 1 | 1 | 0 | 0 | 0 | 0 | 1 | 1 | 0 | 1 | 0 | 0 | 1 | 1 | 0 | 0 | 0.1 | 0.0 |
| 76 | 0 | 1 | 1 | 0 | 0 | 0 | 0 | 1 | 1 | 1 | 1 | 1 | 0 | 0 | 1 | 1 | 0 | 0 | 0.1 | 0.0 |
| 77 | 0 | 1 | 1 | 0 | 0 | 0 | 0 | 1 | 1 | 1 | 1 | 1 | 0 | 0 | 0 | 1 | 0 | 0 | 0.1 | 0.0 |
| 78 | 0 | 1 | 1 | 0 | 0 | 0 | 0 | 1 | 1 | 1 | 0 | 1 | 1 | 0 | 1 | 1 | 0 | 0 | 0.1 | 0.0 |
| 79 | 0 | 1 | 1 | 0 | 0 | 0 | 0 | 1 | 1 | 1 | 0 | 1 | 0 | 0 | 0 | 1 | 0 | 0 | 0.1 | 0.0 |
| 80 | 0 | 1 | 1 | 0 | 0 | 0 | 0 | 1 | 1 | 1 | 0 | 0 | 1 | 0 | 1 | 1 | 0 | 0 | 0.1 | 0.0 |
| 81 | 0 | 1 | 1 | 0 | 0 | 0 | 0 | 1 | 1 | 1 | 0 | 0 | 0 | 1 | 1 | 0 | 0 | 0 | 0.1 | 0.0 |
| 82 | 0 | 1 | 1 | 0 | 0 | 0 | 0 | 1 | 1 | 1 | 0 | 0 | 0 | 0 | 0 | 1 | 0 | 0 | 0.1 | 0.0 |
| 83 | 0 | 1 | 1 | 0 | 0 | 0 | 0 | 0 | 1 | 1 | 1 | 1 | 0 | 0 | 1 | 0 | 0 | 0 | 0.1 | 0.0 |
| 84 | 0 | 1 | 1 | 0 | 0 | 0 | 0 | 0 | 1 | 0 | 0 | 1 | 0 | 0 | 0 | 0 | 0 | 0 | 0.1 | 0.0 |
| 85 | 0 | 1 | 0 | 1 | 0 | 0 | 0 | 1 | 1 | 1 | 0 | 0 | 0 | 0 | 1 | 1 | 0 | 0 | 0.1 | 0.0 |
| 86 | 0 | 1 | 0 | 1 | 0 | 0 | 0 | 1 | 1 | 1 | 0 | 0 | 0 | 0 | 1 | 0 | 0 | 0 | 0.1 | 0.0 |
| 87 | 0 | 1 | 0 | 1 | 0 | 0 | 0 | 0 | 1 | 1 | 0 | 0 | 0 | 0 | 1 | 0 | 0 | 0 | 0.1 | 0.0 |
| 88 | 0 | 1 | 0 | 0 | 0 | 0 | 0 | 1 | 1 | 1 | 1 | 1 | 0 | 0 | 1 | 0 | 0 | 0 | 0.1 | 0.0 |
| 89 | 0 | 1 | 0 | 0 | 0 | 0 | 0 | 1 | 1 | 1 | 0 | 1 | 0 | 1 | 1 | 0 | 0 | 0 | 0.1 | 0.0 |
| 90 | 0 | 1 | 0 | 0 | 0 | 0 | 0 | 1 | 1 | 1 | 0 | 1 | 0 | 0 | 0 | 1 | 0 | 0 | 0.1 | 0.0 |
| 91 | 0 | 1 | 0 | 0 | 0 | 0 | 0 | 1 | 1 | 0 | 0 | 0 | 0 | 0 | 1 | 0 | 0 | 0 | 0.1 | 0.0 |
| 92 | 0 | 0 | 1 | 1 | 0 | 0 | 0 | 1 | 1 | 1 | 0 | 1 | 0 | 1 | 1 | 1 | 0 | 0 | 0.1 | 0.0 |
| 93 | 0 | 0 | 1 | 1 | 0 | 0 | 0 | 1 | 0 | 1 | 1 | 1 | 1 | 1 | 1 | 0 | 0 | 0 | 0.1 | 0.0 |
| 94 | 0 | 0 | 1 | 1 | 0 | 0 | 0 | 1 | 0 | 1 | 1 | 1 | 1 | 0 | 1 | 1 | 0 | 0 | 0.1 | 0.0 |
| 95 | 0 | 0 | 1 | 1 | 0 | 0 | 0 | 1 | 0 | 1 | 0 | 0 | 0 | 1 | 1 | 0 | 0 | 0 | 0.1 | 0.0 |
| 96 | 0 | 0 | 1 | 1 | 0 | 0 | 0 | 1 | 0 | 0 | 1 | 1 | 0 | 0 | 1 | 1 | 0 | 0 | 0.1 | 0.0 |
| 97 | 0 | 0 | 1 | 1 | 0 | 0 | 0 | 1 | 0 | 0 | 0 | 1 | 0 | 0 | 1 | 1 | 0 | 0 | 0.1 | 0.0 |
| 98 | 0 | 0 | 1 | 1 | 0 | 0 | 0 | 0 | 1 | 1 | 0 | 1 | 0 | 0 | 1 | 0 | 0 | 0 | 0.1 | 0.0 |
| 99 | 0 | 0 | 1 | 1 | 0 | 0 | 0 | 0 | 0 | 1 | 0 | 1 | 0 | 0 | 0 | 0 | 0 | 0 | 0.1 | 0.0 |
| 100 | 0 | 0 | 1 | 0 | 0 | 0 | 1 | 1 | 1 | 1 | 1 | 1 | 0 | 1 | 1 | 0 | 0 | 0 | 0.1 | 0.0 |
| 101 | 0 | 0 | 1 | 0 | 0 | 0 | 1 | 1 | 1 | 1 | 0 | 1 | 0 | 1 | 1 | 0 | 0 | 0 | 0.1 | 0.0 |
| 102 | 0 | 0 | 1 | 0 | 0 | 0 | 1 | 1 | 1 | 0 | 1 | 1 | 0 | 1 | 1 | 0 | 0 | 0 | 0.1 | 0.0 |
| 103 | 0 | 0 | 1 | 0 | 0 | 0 | 0 | 1 | 1 | 1 | 1 | 1 | 0 | 1 | 1 | 1 | 0 | 0 | 0.1 | 0.0 |
| 104 | 0 | 0 | 1 | 0 | 0 | 0 | 0 | 1 | 1 | 1 | 0 | 1 | 1 | 0 | 1 | 0 | 0 | 0 | 0.1 | 0.0 |
| 105 | 0 | 0 | 1 | 0 | 0 | 0 | 0 | 1 | 1 | 0 | 0 | 1 | 0 | 1 | 1 | 0 | 0 | 0 | 0.1 | 0.0 |
| 106 | 0 | 0 | 1 | 0 | 0 | 0 | 0 | 1 | 1 | 0 | 0 | 1 | 0 | 0 | 0 | 1 | 0 | 0 | 0.1 | 0.0 |
| 107 | 0 | 0 | 1 | 0 | 0 | 0 | 0 | 1 | 0 | 1 | 1 | 1 | 1 | 0 | 1 | 0 | 0 | 0 | 0.1 | 0.0 |
| 108 | 0 | 0 | 1 | 0 | 0 | 0 | 0 | 1 | 0 | 1 | 0 | 1 | 0 | 0 | 0 | 1 | 0 | 0 | 0.1 | 0.0 |
| 109 | 0 | 0 | 1 | 0 | 0 | 0 | 0 | 1 | 0 | 1 | 0 | 0 | 0 | 1 | 1 | 0 | 0 | 0 | 0.1 | 0.0 |
| 110 | 0 | 0 | 1 | 0 | 0 | 0 | 0 | 1 | 0 | 0 | 1 | 1 | 0 | 1 | 0 | 0 | 0 | 0 | 0.1 | 0.0 |
| 111 | 0 | 0 | 1 | 0 | 0 | 0 | 0 | 1 | 0 | 0 | 0 | 1 | 0 | 1 | 1 | 1 | 0 | 0 | 0.1 | 0.0 |
| 112 | 0 | 0 | 1 | 0 | 0 | 0 | 0 | 0 | 1 | 1 | 1 | 1 | 0 | 0 | 1 | 0 | 0 | 0 | 0.1 | 0.0 |
| 113 | 0 | 0 | 1 | 0 | 0 | 0 | 0 | 0 | 1 | 0 | 1 | 1 | 0 | 0 | 1 | 0 | 0 | 0 | 0.1 | 0.0 |
| 114 | 0 | 0 | 1 | 0 | 0 | 0 | 0 | 0 | 1 | 0 | 0 | 1 | 0 | 0 | 1 | 0 | 0 | 0 | 0.1 | 0.0 |
| 115 | 0 | 0 | 1 | 0 | 0 | 0 | 0 | 0 | 1 | 0 | 0 | 0 | 0 | 0 | 0 | 0 | 0 | 0 | 0.1 | 0.0 |
| 116 | 0 | 0 | 1 | 0 | 0 | 0 | 0 | 0 | 0 | 1 | 1 | 0 | 0 | 0 | 1 | 0 | 0 | 0 | 0.1 | 0.0 |
| 117 | 0 | 0 | 0 | 1 | 0 | 0 | 0 | 1 | 1 | 0 | 0 | 1 | 0 | 0 | 0 | 0 | 0 | 0 | 0.1 | 0.0 |
| 118 | 0 | 0 | 0 | 1 | 0 | 0 | 0 | 1 | 0 | 1 | 0 | 0 | 0 | 0 | 0 | 0 | 0 | 0 | 0.1 | 0.0 |
| 119 | 0 | 0 | 0 | 1 | 0 | 0 | 0 | 1 | 0 | 0 | 0 | 1 | 0 | 1 | 1 | 0 | 0 | 0 | 0.1 | 0.0 |
| 120 | 0 | 0 | 0 | 1 | 0 | 0 | 0 | 0 | 1 | 0 | 0 | 1 | 0 | 0 | 1 | 0 | 0 | 0 | 0.1 | 0.0 |
| 121 | 0 | 0 | 0 | 1 | 0 | 0 | 0 | 0 | 0 | 1 | 0 | 1 | 0 | 0 | 1 | 0 | 0 | 0 | 0.1 | 0.0 |
| 122 | 0 | 0 | 0 | 1 | 0 | 0 | 0 | 0 | 0 | 1 | 0 | 1 | 0 | 0 | 0 | 0 | 0 | 0 | 0.1 | 0.0 |
| 123 | 0 | 0 | 0 | 1 | 0 | 0 | 0 | 0 | 0 | 0 | 1 | 1 | 0 | 0 | 0 | 0 | 0 | 0 | 0.1 | 0.0 |
| 124 | 0 | 0 | 0 | 0 | 0 | 0 | 0 | 1 | 1 | 1 | 0 | 1 | 0 | 1 | 1 | 0 | 0 | 0 | 0.1 | 0.0 |
| 125 | 0 | 0 | 0 | 0 | 0 | 0 | 0 | 1 | 1 | 0 | 0 | 0 | 0 | 0 | 1 | 0 | 0 | 0 | 0.1 | 0.0 |
| 126 | 0 | 0 | 0 | 0 | 0 | 0 | 0 | 1 | 0 | 1 | 0 | 0 | 0 | 0 | 0 | 1 | 0 | 0 | 0.1 | 0.0 |
| 127 | 0 | 0 | 0 | 0 | 0 | 0 | 0 | 1 | 0 | 0 | 0 | 0 | 0 | 1 | 1 | 0 | 0 | 0 | 0.1 | 0.0 |
| 128 | 0 | 0 | 0 | 0 | 0 | 0 | 0 | 1 | 0 | 0 | 0 | 0 | 0 | 0 | 0 | 1 | 0 | 0 | 0.1 | 0.0 |
| 129 | 0 | 0 | 0 | 0 | 0 | 0 | 0 | 0 | 1 | 0 | 1 | 1 | 0 | 0 | 0 | 0 | 0 | 0 | 0.1 | 0.0 |
| 130 | 0 | 0 | 0 | 0 | 0 | 0 | 0 | 0 | 0 | 0 | 0 | 1 | 0 | 0 | 1 | 1 | 0 | 0 | 0.1 | 0.0 |
| 131 | 0 | 0 | 0 | 0 | 0 | 0 | 0 | 0 | 0 | 0 | 0 | 1 | 0 | 0 | 0 | 1 | 0 | 0 | 0.1 | 0.0 |

| | | |
|---|---|---|
| total | 28.7 | 0 |

²Protein code = binary code per line

TABLE 4

| | | | | | | | | | | | | | | | | | | | V1 | V2 |
|---|---|---|---|---|---|---|---|---|---|---|---|---|---|---|---|---|---|---|---|---|
| | | | | | | | | | | | | | | | | | | | No. of | No. of |
| | | | | | | | | | | | | | | | | | | | cells | cells |
| Protein code³ | Proteins [1-18], binary | | | | | | | | | | | | | | | | | | In | In |
| No. | 1 | 2 | 3 | 4 | 5 | 6 | 7 | 8 | 9 | 10 | 11 | 12 | 13 | 14 | 15 | 16 | 17 | 18 | 1/1000 | 1/1000 |
| 1 | 1 | 1 | 1 | 1 | 0 | 0 | 0 | 1 | 1 | 1 | 0 | 0 | 0 | 0 | 1 | 1 | 0 | 0 | 0.0 | 0.6 |
| 2 | 0 | 0 | 1 | 0 | 0 | 0 | 0 | 1 | 0 | 0 | 0 | 1 | 0 | 1 | 0 | 0 | 0 | 0 | 0.0 | 0.6 |
| 3 | 1 | 1 | 1 | 0 | 0 | 0 | 0 | 1 | 1 | 1 | 0 | 0 | 0 | 0 | 1 | 1 | 0 | 0 | 0.0 | 0.3 |
| 4 | 0 | 0 | 1 | 0 | 0 | 0 | 0 | 0 | 0 | 0 | 0 | 1 | 0 | 1 | 0 | 0 | 0 | 0 | 0.0 | 0.3 |
| 5 | 0 | 1 | 1 | 1 | 0 | 0 | 0 | 1 | 1 | 1 | 1 | 1 | 0 | 0 | 1 | 1 | 0 | 0 | 0.0 | 0.2 |
| 6 | 0 | 1 | 1 | 0 | 0 | 0 | 0 | 1 | 1 | 1 | 1 | 1 | 0 | 0 | 0 | 0 | 0 | 0 | 0.0 | 0.2 |
| 7 | 0 | 1 | 0 | 0 | 0 | 0 | 0 | 1 | 1 | 1 | 0 | 0 | 0 | 0 | 0 | 1 | 0 | 0 | 0.0 | 0.2 |
| 8 | 0 | 0 | 1 | 1 | 0 | 0 | 0 | 0 | 0 | 0 | 0 | 0 | 0 | 0 | 0 | 0 | 0 | 0 | 0.0 | 0.2 |
| 9 | 0 | 0 | 1 | 0 | 0 | 0 | 0 | 0 | 0 | 1 | 1 | 1 | 0 | 0 | 0 | 0 | 0 | 0 | 0.0 | 0.2 |
| 10 | 0 | 0 | 0 | 0 | 0 | 0 | 0 | 1 | 1 | 1 | 0 | 0 | 0 | 0 | 1 | 0 | 0 | 0 | 0.0 | 0.2 |
| 11 | 0 | 0 | 0 | 0 | 0 | 0 | 0 | 0 | 0 | 0 | 0 | 0 | 0 | 1 | 1 | 0 | 0 | 0 | 0.0 | 0.2 |
| 12 | 1 | 1 | 1 | 1 | 0 | 0 | 0 | 1 | 1 | 1 | 1 | 1 | 0 | 0 | 1 | 0 | 0 | 0 | 0.0 | 0.1 |
| 13 | 1 | 1 | 1 | 1 | 0 | 0 | 0 | 0 | 1 | 1 | 0 | 0 | 1 | 1 | 1 | 1 | 0 | 0 | 0.0 | 0.1 |
| 14 | 1 | 1 | 1 | 1 | 0 | 0 | 0 | 0 | 1 | 1 | 0 | 0 | 0 | 0 | 1 | 0 | 0 | 0 | 0.0 | 0.1 |
| 15 | 1 | 1 | 0 | 1 | 0 | 0 | 0 | 0 | 1 | 1 | 0 | 0 | 0 | 0 | 1 | 1 | 0 | 0 | 0.0 | 0.1 |
| 16 | 1 | 1 | 0 | 0 | 0 | 0 | 0 | 0 | 1 | 1 | 1 | 0 | 0 | 0 | 1 | 0 | 0 | 0 | 0.0 | 0.1 |
| 17 | 1 | 1 | 0 | 0 | 0 | 0 | 0 | 0 | 1 | 1 | 0 | 0 | 1 | 1 | 1 | 1 | 0 | 0 | 0.0 | 0.1 |
| 18 | 1 | 0 | 1 | 1 | 0 | 0 | 1 | 1 | 1 | 1 | 1 | 1 | 0 | 1 | 1 | 1 | 0 | 0 | 0.0 | 0.1 |
| 19 | 1 | 0 | 1 | 1 | 0 | 0 | 1 | 1 | 0 | 1 | 0 | 1 | 0 | 1 | 1 | 1 | 0 | 0 | 0.0 | 0.1 |
| 20 | 1 | 0 | 1 | 1 | 0 | 0 | 1 | 1 | 0 | 1 | 0 | 0 | 0 | 1 | 1 | 0 | 0 | 0 | 0.0 | 0.1 |
| 21 | 1 | 0 | 1 | 1 | 0 | 0 | 0 | 1 | 1 | 1 | 0 | 1 | 0 | 0 | 1 | 0 | 0 | 0 | 0.0 | 0.1 |
| 22 | 1 | 0 | 0 | 1 | 0 | 0 | 0 | 0 | 1 | 0 | 0 | 0 | 0 | 0 | 1 | 0 | 0 | 0 | 0.0 | 0.1 |
| 23 | 1 | 0 | 0 | 1 | 0 | 0 | 0 | 0 | 0 | 0 | 0 | 1 | 0 | 0 | 1 | 0 | 0 | 0 | 0.0 | 0.1 |
| 24 | 1 | 0 | 0 | 0 | 0 | 0 | 0 | 1 | 0 | 0 | 0 | 1 | 0 | 1 | 1 | 0 | 0 | 0 | 0.0 | 0.1 |
| 25 | 1 | 0 | 0 | 0 | 0 | 0 | 0 | 0 | 0 | 1 | 0 | 1 | 0 | 1 | 1 | 1 | 0 | 0 | 0.0 | 0.1 |
| 26 | 1 | 0 | 0 | 0 | 0 | 0 | 0 | 0 | 0 | 0 | 0 | 1 | 0 | 0 | 1 | 0 | 0 | 0 | 0.0 | 0.1 |
| 27 | 0 | 1 | 1 | 1 | 0 | 0 | 0 | 1 | 1 | 0 | 0 | 0 | 0 | 0 | 1 | 0 | 0 | 0 | 0.0 | 0.1 |
| 28 | 0 | 1 | 1 | 1 | 0 | 0 | 0 | 1 | 0 | 1 | 0 | 1 | 0 | 1 | 1 | 0 | 0 | 0 | 0.0 | 0.1 |
| 29 | 0 | 1 | 1 | 1 | 0 | 0 | 0 | 0 | 1 | 1 | 0 | 1 | 0 | 0 | 1 | 0 | 0 | 0 | 0.0 | 0.1 |
| 30 | 0 | 1 | 1 | 0 | 0 | 0 | 0 | 1 | 1 | 1 | 1 | 1 | 0 | 1 | 1 | 1 | 0 | 0 | 0.0 | 0.1 |
| 31 | 0 | 1 | 1 | 0 | 0 | 0 | 0 | 1 | 1 | 0 | 1 | 1 | 0 | 0 | 1 | 0 | 0 | 0 | 0.0 | 0.1 |
| 32 | 0 | 1 | 1 | 0 | 0 | 0 | 0 | 1 | 1 | 0 | 0 | 1 | 0 | 1 | 1 | 0 | 0 | 0 | 0.0 | 0.1 |
| 33 | 0 | 1 | 1 | 0 | 0 | 0 | 0 | 0 | 1 | 1 | 0 | 1 | 0 | 0 | 1 | 1 | 0 | 0 | 0.0 | 0.1 |
| 34 | 0 | 1 | 1 | 0 | 0 | 0 | 0 | 0 | 1 | 1 | 0 | 0 | 1 | 0 | 0 | 1 | 0 | 0 | 0.0 | 0.1 |
| 35 | 0 | 1 | 1 | 0 | 0 | 0 | 0 | 0 | 1 | 0 | 1 | 1 | 0 | 0 | 0 | 0 | 0 | 0 | 0.0 | 0.1 |
| 36 | 0 | 1 | 0 | 1 | 0 | 0 | 0 | 0 | 1 | 1 | 0 | 0 | 0 | 0 | 1 | 1 | 0 | 0 | 0.0 | 0.1 |
| 37 | 0 | 1 | 0 | 0 | 0 | 0 | 0 | 1 | 1 | 1 | 1 | 1 | 0 | 1 | 1 | 0 | 0 | 0 | 0.0 | 0.1 |
| 38 | 0 | 1 | 0 | 0 | 0 | 0 | 0 | 1 | 1 | 0 | 1 | 1 | 0 | 0 | 1 | 0 | 0 | 0 | 0.0 | 0.1 |
| 39 | 0 | 1 | 0 | 0 | 0 | 0 | 0 | 1 | 1 | 0 | 0 | 0 | 0 | 0 | 0 | 0 | 0 | 0 | 0.0 | 0.1 |
| 40 | 0 | 1 | 0 | 0 | 0 | 0 | 0 | 0 | 1 | 1 | 0 | 1 | 0 | 0 | 0 | 1 | 0 | 0 | 0.0 | 0.1 |
| 41 | 0 | 0 | 1 | 1 | 0 | 0 | 1 | 1 | 0 | 1 | 1 | 1 | 0 | 1 | 1 | 0 | 0 | 0 | 0.0 | 0.1 |
| 42 | 0 | 0 | 1 | 1 | 0 | 0 | 0 | 1 | 1 | 1 | 0 | 0 | 0 | 0 | 1 | 0 | 0 | 0 | 0.0 | 0.1 |
| 43 | 0 | 0 | 1 | 1 | 0 | 0 | 0 | 1 | 1 | 0 | 0 | 1 | 0 | 1 | 1 | 0 | 0 | 0 | 0.0 | 0.1 |
| 44 | 0 | 0 | 1 | 1 | 0 | 0 | 0 | 1 | 0 | 0 | 1 | 0 | 0 | 0 | 0 | 0 | 0 | 0 | 0.0 | 0.1 |
| 45 | 0 | 0 | 1 | 0 | 0 | 0 | 1 | 1 | 0 | 1 | 0 | 1 | 0 | 1 | 1 | 1 | 0 | 0 | 0.0 | 0.1 |
| 46 | 0 | 0 | 1 | 0 | 0 | 0 | 0 | 1 | 1 | 1 | 0 | 1 | 0 | 0 | 1 | 1 | 0 | 0 | 0.0 | 0.1 |
| 47 | 0 | 0 | 1 | 0 | 0 | 0 | 0 | 1 | 1 | 0 | 1 | 0 | 0 | 0 | 0 | 0 | 0 | 0 | 0.0 | 0.1 |
| 48 | 0 | 0 | 1 | 0 | 0 | 0 | 0 | 1 | 0 | 1 | 1 | 1 | 0 | 1 | 0 | 0 | 0 | 0 | 0.0 | 0.1 |
| 49 | 0 | 0 | 1 | 0 | 0 | 0 | 0 | 1 | 0 | 1 | 1 | 0 | 0 | 0 | 0 | 0 | 0 | 0 | 0.0 | 0.1 |
| 50 | 0 | 0 | 1 | 0 | 0 | 0 | 0 | 1 | 0 | 1 | 0 | 1 | 0 | 1 | 0 | 0 | 0 | 0 | 0.0 | 0.1 |
| 51 | 0 | 0 | 1 | 0 | 0 | 0 | 0 | 1 | 0 | 1 | 0 | 0 | 0 | 0 | 0 | 1 | 0 | 0 | 0.0 | 0.1 |
| 52 | 0 | 0 | 1 | 0 | 0 | 0 | 0 | 0 | 1 | 1 | 0 | 0 | 0 | 0 | 1 | 0 | 0 | 0 | 0.0 | 0.1 |
| 53 | 0 | 0 | 0 | 1 | 0 | 0 | 0 | 1 | 0 | 0 | 0 | 0 | 0 | 0 | 1 | 0 | 0 | 0 | 0.0 | 0.1 |
| 54 | 0 | 0 | 0 | 1 | 0 | 0 | 0 | 0 | 1 | 0 | 0 | 0 | 0 | 0 | 1 | 0 | 0 | 0 | 0.0 | 0.1 |
| 55 | 0 | 0 | 0 | 0 | 0 | 0 | 0 | 1 | 1 | 1 | 1 | 1 | 0 | 1 | 1 | 0 | 0 | 0 | 0.0 | 0.1 |
| 56 | 0 | 0 | 0 | 0 | 0 | 0 | 0 | 1 | 1 | 1 | 1 | 1 | 0 | 0 | 0 | 0 | 0 | 0 | 0.0 | 0.1 |
| 57 | 0 | 0 | 0 | 0 | 0 | 0 | 0 | 1 | 1 | 0 | 0 | 1 | 0 | 1 | 1 | 0 | 0 | 0 | 0.0 | 0.1 |
| 58 | 0 | 0 | 0 | 0 | 0 | 0 | 0 | 1 | 0 | 1 | 1 | 1 | 0 | 1 | 1 | 0 | 0 | 0 | 0.0 | 0.1 |
| 59 | 0 | 0 | 0 | 0 | 0 | 0 | 0 | 0 | 0 | 1 | 1 | 1 | 0 | 0 | 0 | 0 | 0 | 0 | 0.0 | 0.1 |
| 60 | 0 | 0 | 0 | 0 | 0 | 0 | 0 | 0 | 0 | 0 | 0 | 0 | 0 | 1 | 0 | 0 | 0 | 0 | 0.0 | 0.1 |

| | | |
|---|---|---|
| total | 0.0 | 9.1 |

³Protein code = binary code per line

1. Utilization of at least one aminopeptidase inhibitor for the production of a medicament used in the treatment of tumor diseases and/or immune diseases, whereby the at least one aminopeptidase inhibitor causes blocking of polarization of invasive human or animal tumor cells and/or immune cells by modifying at least one surface protein CD13 as member of a protein network on the surface of the tumor cells and/or immune cells, whereby the protein network comprises up to 30 surface proteins from a group consisting of
```
  1. CD4
  2. CD8
  3. HLA-DR
  4. HLA-DQ
  5. CD3
  6. CD26
  7. CD38
  8. CD45RA
  9. CD16
10. CD57
11. CD56
12. CD7
13. CD54
14. CD58
15. CD138
16. CD13
17. CD62L
18. CD71
19. CD11b
20. CD36
21. CD29
22. CD49d
23. CD18
24. CD49f
25. CD19
26. CD2
27. CD20
28. CD10
29. CD44
30. CD80.
```

2. The utilization as claimed in claim 1
characterized in that
said at least one aminopeptidase inhibitor is an aminopeptidase inhibitor of the homophtalimide type and/or actinonin and/or bestatin, and/or an antibody, in particular a monoclonal antibody, against one of said surface proteins.

3. The utilization as claimed in claim 1
characterized in that
said immune diseases are autoimmune diseases or rejections of transplanted organs or allergies, in particular allergies of the respiratory tract.

4. The utilization as claimed in claim 1 or 2
characterized in that
for producing said medicament, at least one additional inhibitor is used which inhibits at least one surface protein that is not an aminopeptidase.

5. The utilization as claimed in claim 1
characterized in that
at least one aminopeptidase inhibitor and/or at least one additional inhibitor causes a modification of at least one surface protein of said tumor cells and/or immune cells which surface protein is responsible for adhesion to endothelial cells and/or extracellular structures, in particular organ-specific endothelial cells and/or organ-specific extracellular structures.

6. The utilization as claimed in claim 1
characterized in that
at least one aminopeptidase inhibitor and/or at least one additional inhibitor will cause modification of the adhesive functions of endothelial cells.

7. The utilization as claimed in claim 1
characterized in that
the expression of at least one surface protein, in particular of an adhesion molecule, may be influenced by means of at least one aminopeptidase inhibitor and/or at least one additional inhibitor.

8. A pharmaceutical preparation which can be produced using at least one aminopeptidase inhibitor or a combination of at least one aminopeptidase inhibitor and at least one additional inhibitor as claimed in claims 1 to 7.

9. A method for identifying at least one aminopeptidase inhibitor which causes blocking of polarization of invasive human or animal tumor cells and/or immune cells, comprising:
a) detecting surface protein combinations of a protein network which are on the surface of the untreated tumor Hs cells and/or immune cells, whereby the protein network comprises up to 30 surface proteins from a group consisting of
```
  1. CD4
  2. CD8
  3. HLA-DR
  4. HLA-DQ
  5. CD3
  6. CD26
  7. CD38
  8. CD45RA
  9. CD16
10. CD57
11. CD56
12. CD7
13. CD54
14. CD58
15. CD138
16. CD13
17. CD62L
18. CD71
19. CD11b
20. CD36
21. CD29
22. CD49d
23. CD18
24. CD49f
25. CD19
26. CD2
27. CD20
28. CD10
29. CD44
30. CD80;
```
b) treating said or similar tumor cells and/or immune cells with at least one aminopeptidase inhibitor;
c) detecting said surface protein combinations of the protein network which are on the surface of the treated tumor cells and/or immune cells; and
d) comparing the surface protein combinations detected in steps a) and c), whereby the at least one aminopeptidase inhibitor, if there is a divergence of the surface protein combinations detected in step a) from the surface protein combinations detected in step c) in that there is at least one modification of surface protein CD13, will cause blocking of polarization of said tumor cells and/or immune cells.

10. The method as claimed in claim 9
characterized in that
said method includes a further step, following step d), in which the at least one aminopeptidase inhibitor identified in step d) is added to at least one polarizing tumor cell and/or immune cell, and the further development of the at least one polarizing tumor cell and/or immune cell is detected.

11. The method as claimed in one of claims 9 or 10
characterized in that
said method includes a further step, following step d), in which any binding of the untreated tumor cells and/or immune cells to organ-specific endothelial cells and/or to organ-specific extracellular structures is detected, in which any binding of the tumor cells and/or immune cells treated with the at least one aminopeptidase inhibitor identified in step d) to the organ-specific endothelial cells and/or to the organ-specific extracellular structures is detected, and in which the detected bindings are compared.

12. A method for identifying at least one inhibitor which—in combination with at least one aminopeptidase inhibitor—will cause blocking of polarization of invasive human or animal tumor cells and/or immune cells, comprising:
a) detecting surface protein combinations of a protein network which are on the surface of the untreated tumor cells and/or immune cells, whereby the protein network comprises up to 30 surface proteins from a group consisting of
```
  1. CD4
  2. CD8
  3. HLA-DR
  4. HLA-DQ
  5. CD3
  6. CD26
  7. CD38
  8. CD45RA
  9. CD16
10. CD57
11. CD56
12. CD7
13. CD54
14. CD58
15. CD138
16. CD13
17. CD62L
18. CD71
19. CD11b
20. CD36
21. CD29
22. CD49d
23. CD18
24. CD49f
25. CD19
26. CD2
27. CD20
28. CD10
29. CD44
30. CD80;
```
b) treating said or similar tumor cells and/or immune cells with at least one potential inhibitor which is not directed against an aminopeptidase;
c) detecting the surface protein combinations of the protein network which are on the surface of the treated tumor cells and/or immune cells; and
d) comparing the surface protein combinations detected in steps a) and c), whereby the at least one inhibitor, if there is a divergence of the surface protein combinations detected in step a) from the surface protein combinations detected in step c) in that there is at least one modification of a surface protein, will be suitable for blocking polarization of said tumor cells and/or immune cells.

13. The method as claimed in claim 12
characterized in that
said or the similar tumor cells and/or immune cells are also treated with at least one aminopeptidase inhibitor in step b), with the combination of the at least one inhibitor and the at least one aminopeptidase inhibitor, if there is a divergence of the surface protein combinations detected in step a) from the surface protein combinations detected in step c) in that there is at least one modification of a surface protein CD13, will cause blocking of polarization of the tumor cells and/or immune cells.

14. The method as claimed in one of claims 12 or 13
characterized in that
said method includes a further step, following step d), in which the at least one aminopeptidase inhibitor identified in step d) or a combination of the at least one inhibitor identified in step d) and at least one aminopeptidase inhibitor is added to at least one polarizing tumor cell and/or immune cell, and the further development of the at least one polarizing tumor cell and/or immune cell is detected.

15. The method as claimed in one of claims 12 to 14
characterized in that
said method includes a further step, following step d), in which any binding of the untreated tumor cells and/or immune cells to organ-specific endothelial cells and/or to organ-specific extracellular structures is detected, in which any binding of the tumor cells and/or immune cells treated with the at least one inhibitor identified in step d) or with a combination of the at least one inhibitor identified in step d) and at least one aminopeptidase inhibitor to the organ-specific endothelial cells and/or to the organ-specific extracellular structures is detected, and in which the detected bindings are compared.
